(19) United States
(12) Patent Application Publication
(10) Pub. No.: US 2021/0369917 A1
(43) Pub. Date: Dec. 2, 2021

(54) MODULAR BIOFABRICATION PLATFORM FOR DIVERSE TISSUE ENGINEERING APPLICATIONS AND RELATED METHOD THEREOF

(71) Applicant: University of Virginia Patent Foundation, Charlottesville, VA (US)

(72) Inventors: George Christ, Crozet, VA (US); Poonam Sharma, Charlottesville, VA (US); William Hess, Afton, VA (US); Rachel Bour, Charlottesville, VA (US)

(73) Assignee: University of Virginia Patent Foundation, Charlottesville, VA (US)

(21) Appl. No.: 17/282,117

(22) PCT Filed: Oct. 4, 2019
(86) PCT No.: PCT/US2019/054744

§ 371 Date: Apr. 1, 2021

Related U.S. Application Data
(60) Provisional application No. 62/741,215, filed on Oct. 4, 2018.

(51) Int. Cl. A61L 27/38; A61L 27/36; A61L 27/34; C12M 3/00; B33Y 10/00; B33Y 30/00; B33Y 80/00; B29C 64/106; B29C 64/245

(52) U.S. Cl. CPC A61L 27/38; A61L 27/3691; A61L 27/34; C12M 21/08; B33Y 10/00; B29L 2031/753; B33Y 80/00; B29C 64/106; B29C 64/245; B29K 2005/00; B33Y 30/00

(57) ABSTRACT

System and method of bioprinting used to enable automated fabrication of various constructs with high reproducibility and scalability, while reducing costs and production timelines. The bioprinting applications provides a critical component to the further enrichment the overall biomanufacturing paradigm. The biofabrication of sheet-like implantable constructs and other construct types with cells deposited on both sides—a process that may be both scaffold and cell type agnostic, and furthermore, is amenable to many additional tissue engineering applications beyond skeletal muscle.

### RELATED APPLICATIONS

The present application claims benefit of priority under 35 U.S.C § 119 (e) from U.S. Provisional Application Ser. No. 62/741,215, filed Oct. 4, 2018, entitled “Modular Biofabrication Platform for Diverse Tissue Engineering Applications and Related Method Thereof”; the disclosure of which is hereby incorporated by reference herein in its entirety.

The present application is related to International Patent Application Serial No. PCT/US2016/051948, entitled “BIOREACTOR AND RESEEDING CHAMBER SYSTEM AND RELATED METHODS THEREOF”, filed Sep. 15, 2016; Publication No. WO 2017/048961, Mar. 23, 2017; the disclosure of which is hereby incorporated by reference herein in its entirety.

The present application is related to International Patent Application Serial No. PCT/US2017/045299, entitled “BIOREACTOR CONTROLLER DEVICE AND RELATED METHOD THEREOF”, filed Aug. 3, 2017; Publication No. WO 2018/027033, Feb. 8, 2018; the disclosure of which is hereby incorporated by reference herein in its entirety.

### FIELD OF INVENTION

This invention relates to modular biofabrication platform for diverse tissue engineering applications. More particularly, this invention is directed to biomanufacturing enabled by bioprinting.

### BACKGROUND

Volumetric muscle loss (VML) resulting from traumatic injury and disease, and VML-like congenital and genetic conditions such as cleft lip/palate, are common in both the military and civilian populations^{1,2}. By definition, such injuries and conditions exceed the considerable endogenous regenerative capacity of skeletal muscle and result in permanent cosmetic and functional deficits². Despite significant advances in surgical procedures, VML treatment often requires multiple surgical interventions with generally poor cosmetic and functional outcomes, as current treatments for VML and craniofacial defects do not significantly promote regeneration of missing muscle tissue. Surgical treatments include skin grafts and autologous muscle flaps². Utilizing autologous muscle from the patient poses the risk of donor site morbidity and relies on the availability of sufficient muscle for transfer^{3,4}. Furthermore, there is the possibility of muscle flap failure³. Unfortunately, the most devastating and persistent cosmetic and functional deficits resulting from traumatic VML in service members and civilians cannot be solved with existing reconstructive procedures, and are a major source of long-term disability¹.

With a high occurrence of VML injuries and a lack of treatment options that address the incurred functional and cosmetic deficits, there is a clinical need for additional therapies. There is no current standard of care for VML injury that yields satisfactory functional outcomes, nor any biologic or combination product (of which the present inventor is aware of) that has received Food and Drug Administration (FDA) approval for VML repair. Despite some encouraging initial clinical results from implantation of decellularized extracellular matrices (dECM) for treatment of VML in patients, it is clear that there is still significant room for therapeutic improvement⁵⁻⁷. As such, continued development of tissue engineering and regenerative medicine technologies/products has enormous potential to provide a therapeutic solution for VML, and this opportunity has spurred robust preclinical activity⁸⁻¹⁵. Finally, as discussed in great detail herein, there are still significant challenges remaining with respect to biomanufacturing these products.

Decellularized Extracellular Matrix (dECM): A “Ready-Made” Scaffolding Material

Methods that have been employed for skeletal muscle repair include implantation of acellular scaffolds¹⁶⁻²², minced muscle grafts²³, cell-laden scaffolds^{11-13,24-26}, and bioprinted constructs²⁷. While a variety of naturally derived and chemically synthesized scaffolding materials have been explored, one of the more promising materials for clinical translation is dECM, which will be one of the aspects of the various embodiments of the present invention. The extracellular matrices (ECM) is critical to tissue structure and function and plays an important role in cell signaling for migration, proliferation, and differentiation²⁸⁻³¹. However, natural ECM has an extremely complex structure of proteins (such as collagen, laminin, and fibronectin) and polysaccharides (particularly glycosaminoglycans, or GAGs, such as hyaluronic acid)^{28,29,32,33}. This complex structure of ECM is difficult to mimic in engineering²⁹, and the use of biologically sourced materials provides an effective method for capturing this complexity. The field of tissue engineering has made significant strides in harnessing the inherent complexity of the ECM itself by establishing methods for using dECM in tissue regeneration³²⁻³⁶. The promise of dECM as a material in tissue engineering extends beyond its complex structure and there is evidence that dECM retains bound growth factors such as vascular endothelial growth factor (VEGF) which could be beneficial in the context of tissue regeneration^{30,37,38}. Importantly, several sources of dECM have been FDA-approved as implantable devices, including porcine small intestine submucosa (SIS), porcine urinary bladder, human/porcine/bovine dermis, and porcine heart valves^{29,32,34}. Additionally, these materials are abundantly available from porcine sources and fortunately do not illicit a harmful immune response, as components of the ECM are highly conserved across species³⁹.

In the context of preclinical studies for repair of VML, decellularized ECM derived directly from skeletal muscle explants has also been evaluated^{18,22}. However, regardless of the origin of the dECM, while acellular repairs can restore aspects of muscle volume and morphology, they do not promote appreciable muscle fiber regeneration when compared to scaffolds that include a cellular component. This is true even when some functional recovery is observed, and demonstrates the importance of including a cellular component^{13,23,24}. Current methods of combining cells and dECM in a therapy to create a microenvironment that is more favorable for endogenous skeletal muscle regeneration and functional recovery after VML injury have been explored^{20,24,26}. One such current method is described below.

### The Current Tissued Engineered Muscle Repair (TEMR) Biofabrication Process

Tissued engineered muscle repair (TEMR) is an autologous implantable construct capable of volume reconstitution and restoration of clinically relevant force/tension following VML injury in biologically relevant rodent models^{8,9,11-15,26}. The manual biomanufacturing process for the TEMR construct has been published¹¹⁻¹⁴, and is shown generally in the top portion of FIG. 1. This current technology combines muscle derived progenitor cells (MPCs) with a porcine-derived bladder acellular matrix (BAM). The selection of the BAM scaffold for the first generation TEMR technology was based on the following design criteria: (1) biocompatible collagen-based scaffold, (2) biomechanical characteristics suitable for bioreactor preconditioning, (3) sufficient strength for suture retention following implantation in vivo, and (4) favorable biodegradation following implantation in vivo. The BAM scaffold is derived from porcine bladders that are decellularized in a series of detergent solutions, followed by the isolation of the lamina propria layer from the bladder, as previously described¹¹.

Briefly, the current TEMR construct is created by seeding approximately 1×10⁶ muscle progenitor cells (MPCs)/cm² onto each side of a BAM scaffold, followed by 10 days of cell proliferation and differentiation, and then 5-7 days of bioreactor preconditioning, in vitro (i.e., 10% cyclic mechanical stretch, 3 times per minute for the first 5 minutes of every hour). Following this conditioning and maturation period, the TEMR construct exhibits a largely differentiated cellular morphology consisting primarily of myoblasts and myotubes. The entire manual TEMR manufacturing process takes 12 days prior to bioreactor preconditioning, as follows: 2 days of manual seeding (1 day per side); 3 days proliferation and 7 days of differentiation. Implantation of TEMR at the site of VML injury in the present inventor's biologically relevant rodent models can restore clinically relevant force/tension (60-90% functional recovery) within 2-3 months of implantation, providing important proof of concept^{11-15,26}. The present inventor's most recent publication indicates that the size of the injuries envisioned as currently amenable to treatment via TEMR implantation (≈2 cm²) scale well to the present inventor's currently proposed indication for secondary revision of unilateral cleft lip in patients²⁶. With current approaches, sufficient autologous cells for creation of the TEMR construct for this purpose can likely be obtained from a biopsy of ≈1000 mg, perhaps less, of donor leg muscle. Of note, such constructs would also be applicable to the repair of some muscles in the hand and shoulder.

An aspect of an embodiment of the present invention bioprinting methods and related systems hold promise for addressing biomanufacturing challenges associated with scale-up for clinical translation of this technology. These challenges extend beyond the context of VML and the TEMR construct specifically, but the TEMR will be used for the purpose as a model to illustrate these points.

### SUMMARY OF ASPECTS OF VARIOUS EMBODIMENTS OF THE INVENTION

As mentioned above, the following patents, patent applications and patent application publications as listed below are related to aspects of embodiments of the present invention and are hereby incorporated by reference in their entirety herein. The bioreactor related systems, bioreactor related devices, bioreactor methods, bioreactor controllers, methods for bioreactor controllers, and non-transitory computer readable medium to execute a method for a bioreactor controller are considered part of the present invention, and may be employed within the context of the invention.

a. International Patent Application Serial No. PCT/US2016/051948, entitled “BIOREACTOR AND RESEEDING CHAMBER SYSTEM AND RELATED METHODS THEREOF”, filed Sep. 15, 2016; Publication No. WO 2017/048961, Mar. 23, 2017; the disclosure of which is hereby incorporated by reference herein in its entirety.

b. U.S. Utility patent application Ser. No. 15/760,009, entitled “BIOREACTOR AND RESEEDING CHAMBER SYSTEM AND RELATED METHODS THEREOF”, filed Mar. 14, 2018; Publication No. US-2018-0265831-A1, Sep. 20, 2018.

c. International Patent Application Serial No. PCT/US2017/045299, entitled “BIOREACTOR CONTROLLER DEVICE AND RELATED METHOD THEREOF”, filed Aug. 3, 2017; Publication No. WO 2018/027033, Feb. 8, 2018; the disclosure of which is hereby incorporated by reference herein in its entirety.

d. U.S. Utility patent application Ser. No. 16/322,691 to Christ, et al, “Bioreactor Controller Device and Related Method Thereof”, Feb. 1, 2019.

An aspect of an embodiment of the present invention provides, among other things, a bioprinting method, wherein the method may comprise: disposing a scaffold onto a bioassembly device; disposing said bioassembly device, with said scaffold, onto a bioprinter; bioprinting onto a first side of said scaffold or both said first side and a second side of said scaffold, which is disposed on said bioassembly device that is disposed on said bioprinter; transferring said bioprinted scaffold, which is disposed on said bioassembly device, onto a bioreactor; and creating tissue engineered construct while said bioprinted scaffold remains on said bioassembly device and in said bioreactor.

An aspect of an embodiment of the present invention provides, among other things, a bioassembly device for use with a bioprinter, wherein said device may comprise: a top portion and a bottom portion that are configured to secure a scaffold there between while said bioprinter performs bioprinting onto a first side of said scaffold or both said first side and a second side of said scaffold.

An aspect of an embodiment of the present invention provides, among other things, a bioprinting system, where the system may comprise: a designated area configured for receiving a bioassembly device, which includes a scaffold disposed in said bioassembly device; and a print head configured for bioprinting onto a first side of said scaffold or both said first side and a second side of said scaffold, while said bioassembly device is in said designated area of said bioprinting system.

An aspect of an embodiment of the present invention provides, among other things, a system, method, and computer readable medium of bioprinting that is used to enable automated fabrication of various constructs with high reproducibility and scalability, while reducing costs and production timelines. The bioprinting applications provides a critical component to the further enrichment the overall biomanufacturing paradigm. The biofabrication of sheet-like implantable constructs and other construct types and geometrical structures with cells deposited on both sides—a process that may be both scaffold and cell type agnostic, and furthermore, is amenable to many additional tissue engineering applications beyond skeletal muscle.

An aspect of an embodiment of the present invention provides, among other things, bioprinting on sheet-based scaffolds applied to the creation of implantable tissue engineered constructs with potentially diverse clinical applications. As a non-limiting example, tissue engineered muscle repair (TEMR) provides an aspect of an embodiment for illustrative purposes and serves as a representative testbed; and the present invention should not be construed to be limited thereto.

An aspect of an embodiment of the present invention may include pre-clinical therapies for VML repair, with an emphasis on those which utilize dECM, and addresses the need for advanced biomanufacturing enabled by bioprinting. In this context, an aspect of an embodiment of the present invention provides, among other things, a non-classical bioprinting method, system, and a focus of applying it to a representative skeletal muscle repair technology. Also provided herein are preliminary data that highlight the manufacturing challenges addressed by this subset of bioprinting applications. Additionally, other aspects of embodiments will show, among other things, how success in this realm may be more broadly applied to other tissue engineering applications.

### The Need for Advanced Biomanufacturing: Applications of Bioprinting

Here, the terms biomanufacturing and biofabrication are used interchangeably, and both refer to the process of creating a biological product, including but not limited to the use of bioprinting and bioassembly-type technologies to structure cells and materials⁴⁰. More specifically, creation of affordable and scalable tissue engineered products will require simultaneously reducing production time and manufacturing costs while enabling scaling. In this regard, bioprinting can not only be used to produce complex, three dimensional structures, but also as a technology that facilitates the automated manufacturing of cell-dense constructs⁴¹⁻⁴⁴ in a manner that can meet the regulatory requirements of a biomanufacturing process.

An aspect of an embodiment of the present invention shall provide, among other things, a critical role, which bioprinting shall play in the tissue engineering/regenerative medicine space. Specifically, for example, an aspect of an embodiment of the present invention provides, among other things, a technique, method, and system that utilizes bioprinting and sheet-based biofabrication processes. This hybrid biofabrication method is conceptually depicted in FIG. 2, and the benefits of implementation include, but not limited thereto, increasing automation, reproducibility, efficiency, as well as scaling of both research grade and clinical tissue engineered products. In this context, the TEMR technology is applicable as a non-limiting model product for developing this system. Specifically, for example, an aspect of an embodiment of the present invention provides, among other things, bioprinting to directly deposit cells onto scaffolds (comprised of dECM or other materials)—and wherein one of the primary purposes of the scaffold is to provide a biodegradable cell delivery vehicle. This is one of the key distinctions from the approach taken by others, as a goal of an aspect of an embodiment of the present invention TEMR technology biomanufacturing platform is not to provide functional muscle for implantation, but rather to biomanufacture an implantable construct that creates an enhanced microenvironment for improved muscle repair and regeneration in vivo.

TEMR provides a particularly relevant technology for considering the specific challenges, progress, and biomanufacturing potential of bioprinting, as an Investigational New Drug (IND) application that has been submitted by the present inventor to the FDA for the use of this technology in a pilot clinical study for secondary revision of cleft lip. Even at such an early stage in the clinical development cycle of this technology, it is worth considering how advanced biomanufacturing methods could impact clinical translation. In that regard, the novel biofabrication system and method discussed herein has the potential to provide a platform not only for development of implantable skeletal muscle repair technologies, but for a range of additional clinical applications as well—as will be discussed in more detail herein.

An aspect of an embodiment of the present invention bioprinting provides, among other things, a vast potential to enhance the development, manufacturing and scalability of tissue engineering and regenerative medicine technologies for a variety of research and clinical applications. The possibilities range in sophistication from the creation of the complex 3D tissue architectures required for biofabrication of solid organs, to the production of organoids for in vitro investigations. An aspect of an embodiment includes various roles for the use of bioprinting. For example, an aspect of an embodiment includes utilizing bioprinting to automate biomanufacturing of simpler tissue structures, such as the uniform deposition of (mono) layers of progenitor cells on sheet-like decellularized extracellular matrices (dECM). In this scenario, dECM provides a biodegradable cell-delivery matrix for creation of enhanced regenerative microenvironments following in vivo implantation. In fact, as discussed above, previous work by the present inventor has demonstrated that inclusion of muscle progenitor cells on a porcine bladder acellular matrix (BAM) for treatment of rodent volumetric muscle loss (VML) injuries significantly improved tissue repair, volume reconstitution, and functional outcomes. The present inventor refers to this implantable technology platform as tissue engineered muscle repair (TEMR). An aspect of an embodiment of the present invention provides for, among other things, bioprinting the automated fabrication of TEMR constructs with high reproducibility and scalability, while reducing costs and production timelines. The present inventor submits that such bioprinting applications are a critical component to the further enrichment of the overall biomanufacturing paradigm. In particular, for biofabrication of sheet-like implantable constructs with cells deposited on both sides—a process that is both scaffold and cell type agnostic, and furthermore, is amenable to many additional tissue engineering applications beyond skeletal muscle.

Moreover, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The aspects of embodiments of the invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 provides a schematic depiction of the TEMR creation process by traditional methods and provides an aspect of an embodiment of the present invention bioprinting process (and related system and device).

FIG. 2 provides a summary of benefits and advantages of aspects of illustrative embodiments.

FIGS. 3A-3C provides a schematic illustration of aspects of various embodiments of the bioprinting system and related method.

FIG. 4A provide the micrographic depiction that is representative of a composite image to demonstrate the reproducible cell coverage at a lower cell density for an embodiment of the bioprinting system.

FIG. 4B provide the graphical depiction to demonstrate the coverage across multiple points for an embodiment of the bioprinting system.

FIG. 4C provide the micrographic depiction comparing manual seeding to the seeding of an embodiment of the bioprinting.

FIG. 4D provide the graphical depiction comparing manual seeding to the seeding of an embodiment of the bioprinting system.

FIGS. 5A-5D provide the micrographic depictions comparing an initial application of dECM bioprinting to other relevant cell types.

FIGS. 6A-6D provide the photographic and schematic illustrations of respective embodiments of the biofabrication systems and related processes.

FIGS. 7A, 7B, 7D, and 7E provide the micrographic depictions comparing cell viability twenty four hours after printing for respective cell types.

FIGS. 7C and 7F provide the graphical depictions comparing cell viability for respective cell types.

FIGS. 8A-8E provide photographic and micrographic depictions illustrating a workflow of an aspect of embodiment of the biofabrication process for creating next-general TEMR construct with human muscle progenitor cells (MPCs).

FIGS. 9A and 9B schematically illustrate an exploded view and assembled view, respectively, depicting a prototype for the bioassembly device holding a BAM scaffold and functioning as a seeding chamber.

### DETAILED DESCRIPTION OF ASPECTS OF EXEMPLARY EMBODIMENTS

### Materials and Methods

### Bioprinted TEMR Methodology

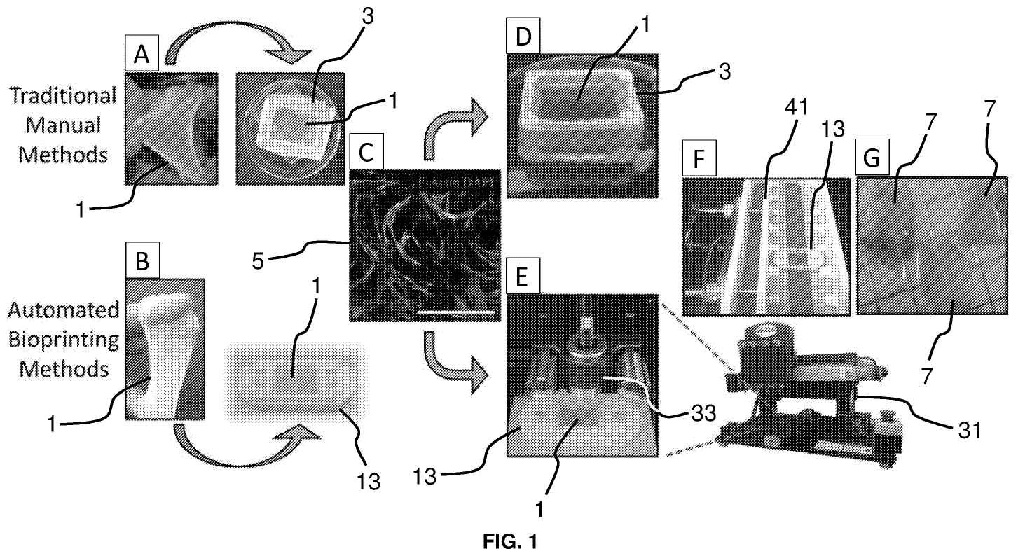

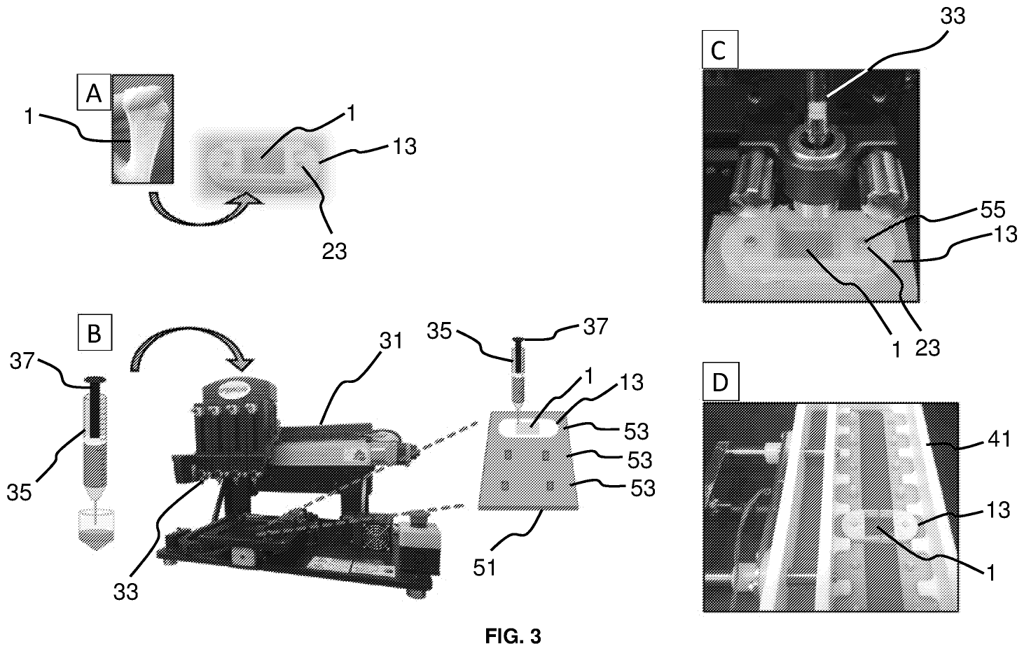

As shown substantially in the bottom portion of FIG. 1, and in further detail in FIG. 3, an aspect of an embodiment of the present invention method (and related system) for TEMR biomanufacturing utilizes a printer, such as the Organovo NovoGen® 3D bioprinter for cell seeding. It should be appreciated that other printer types may be utilized as well. This particular printer is an extrusion-based printer that uses Hamilton syringes and exerts mechanical force on the plunger of the syringe to extrude the bioink through the needle. In an embodiment, the printer is programmed to deposit cells over the surface of the BAM—thus automating the cell seeding process for TEMR biomanufacturing. In an embodiment, the bioink may be a 2% gel containing the skeletal muscle progenitor cells. In this embodiment, hyaluronic acid (HA) was chosen because it is a well-studied polysaccharide, naturally found in the extracellular matrix, and has long been implicated in tissue regeneration⁴⁵⁻⁴⁷. Other biologically derived materials commonly used as bioinks include gelatin⁴⁸⁻⁵⁰, alginate⁵⁰, fibrinogen^{48,49}, and collagen⁵¹, as well as other biopolymers. In applications for skeletal muscle bioprinting, work by Atala and colleagues features a bioink consisting of a combination of fibrinogen, gelatin, and hyaluronic acid^{27,48,52}. Many groups have also developed methods for directly incorporating dECM into bioinks^{53,54}. However, for the purposes of TEMR, the benefits of dECM are harnessed through the BAM scaffold substrate rather than the bioink.

After deciding to use HA as the bioink in this system, printability of HA gel was assessed. Several different weight percentages of HA ranging from 0.5% to 3% were qualitatively assessed (data not shown) and 2% HA by weight was determined to be the optimal formulation for the purposes of this project due to reasonable shape retention, ease of syringe loading, and reliable deposition. It should be appreciated that other levels of percent HA by weight may be implemented as desired or required.

The BAM scaffold for the bioprinted TEMR can be prepared in the same manner as an aspect of an embodiment of the present invention TEMR manufacturing methods described above, and in previously published work¹¹. Both the cell-rich bioink, and the ECM-derived BAM substrate onto which the cells are deposited, play a supportive role during the maturation of a layer of tissue. In this scenario, the bioink serves only to control uniform high-density cell deposition across the entire area of the dECM scaffold (FIG. 1).

FIG. 1 provides a schematic depiction of the TEMR creation process by traditional methods and also provides an aspect of an embodiment of the present invention bioprinting process (and related system and device).

### Traditional Process and Device:

By traditional manual methods, the process requires a total time of 15-17 days. Referring to FIG. 1A, provided is the BAM preparation by traditional methods—the BAM 1 is draped over a mold 3, such as a silicon mold. Other types of scaffolds or matrixes may be used other than the BAM. Referring to FIG. 1C, shown as a micrographic depiction, as provided in either process of FIG. 1, may be isolated skeletal muscle progenitor cells 5 (provided a scale bar=1000 μm). Referring to FIG. 1D, the isolated skeletal muscle progenitor cells 5 are seeded manually at 1×10⁶ cells/cm² onto each side of the BAM 1. These constructs, referring to FIG. 1D, are cultured for 10 days prior to bioreactor preconditioning. Referring to FIG. 1F, the construct from the manual process must be removed from the silicone mold 3, draped, and clamped into the bioreactor 41 for preconditioning and alignment of the differentiating myotubes. Referring to FIG. 1G, provided is a photographic depiction of a completed TEMR construct 7 ready for implantation into a rodent VML injury model (as illustrated here, the completed constructs 7 are created by traditional methods).

### Aspect of an Embodiment of the Present Invention Process and System:

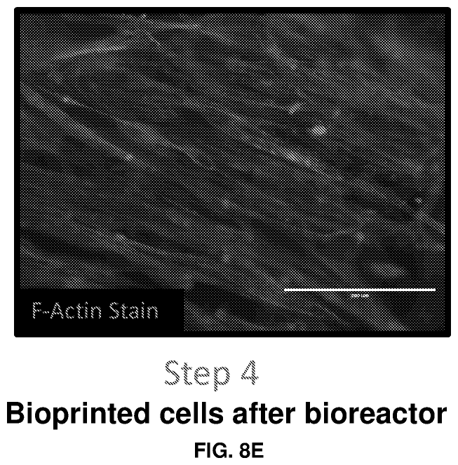

Referring to FIG. 1B, in an aspect of an embodiment of the present invention, in preparation for bioprinting, the BAM scaffold 1 is draped over a specially designed holder as represented by the bioassembly device 13. Referring to FIG. 1C, shown as a micrographic depiction, as provided in either process of FIG. 1 may be isolated skeletal muscle progenitor cells 5 (provided a scale bar=1000 μm). Referring to FIG. 1E, in an aspect of an embodiment of the present invention, by automated methods, using a bioprinter 31 and print head 33 the isolated skeletal muscle progenitor cells are bioprinted in hyaluronic acid gel at a density as low as 1.4×10⁵ cells/cm² onto BAM scaffold 1. It is noted that no proliferation period is required, as a confluent monolayer is present 24 hours after printing the second side of the BAM scaffold 1. Referring to FIG. 1F, the bioprinted construct and holder (bioassembly device 13) can be directly placed into the bioreactor 41 without manual manipulation of the BAM 1. Although FIGS. 1F-1G are merely intended to be a conceptual representative as the items are derived from an experimental traditional process for purpose of discussion. FIGS. 1F-1G may not necessarily be construed as a specific embodiment of the present invention. In contrast, FIG. 8D, which shall be discussed below, illustrates a photographic depiction of an update of an aspect of an embodiment of the present invention bioassembly device 13 in a bioreactor 41.

FIG. 8 provides a schematic illustration of workflow of an aspect of embodiment of the present invention biofabrication process for creating next-generation TEMR construct with human muscle progenitor cells (MPCs).

As generally reflected in FIG. 8A, Step 1 may include a scaffold 1 that is draped on the uniquely designed modular holder or scaffold holder referred to as the bioassembly device 13. This bioassembly device 13 is uniquely designed to fit in the bioprinter 41 for double sided printing of up to, but not limited thereto, three constructs at a time, for example. Moreover, if the capacity and real estate were increased then more than three bioassembly devices may be effected/implemented. Software code or machine instructions is written to print cells (in this case MPCs) onto a specified region of the scaffold 1. Modifications to code(s) or machine instructions and the bioprinter 31 may be implemented to permit this process as desired or required.

As generally reflected in FIG. 8B, Step 2 may include a high density of cells is directly printed onto the scaffold 1 of the bioassembly device 13 by a printer 31 having a print head 33. In an embodiment, the printer may be a three-dimensional (3D) printer. Moreover, if the capacity and real estate were increased and/or the size of the bioassembly 13 decreased then more than three bioassembly devices may be effected/implemented.

Referring to FIG. 8C, shown as a micrographic depiction, is the confluent monolayer 24 hours after printing. It is noted that 24 hours after the construct was bioprinted, the constructs were imaged and stained for DAPI and Actin. For instance, DAPI (4′,6-diamidino-2-phenylindole) is a blue-fluorescent DNA stain.

As generally reflected in FIG. 8D, Step 3 may include whereby the bioprinted constructs are removed from the bioprinter 31 and placed in the bioreactor 41 for incubation and/or automated stretching (cyclic and/or static). In an embodiment, the bioreactor 41 can be programmed to provide cyclic or static stretch, which is known to facilitate differentiation and alignment of the MPCs, for example.

Referring to FIG. 8E, shown as a micrographic depiction, is Step 4 includes that upon completion of bioreactor incubation/preconditioning, the bioprinted constructs are removed from the bioreactor and ready for use/implantation/transportation. The constructs were imaged and stained for DAPI and Actin.

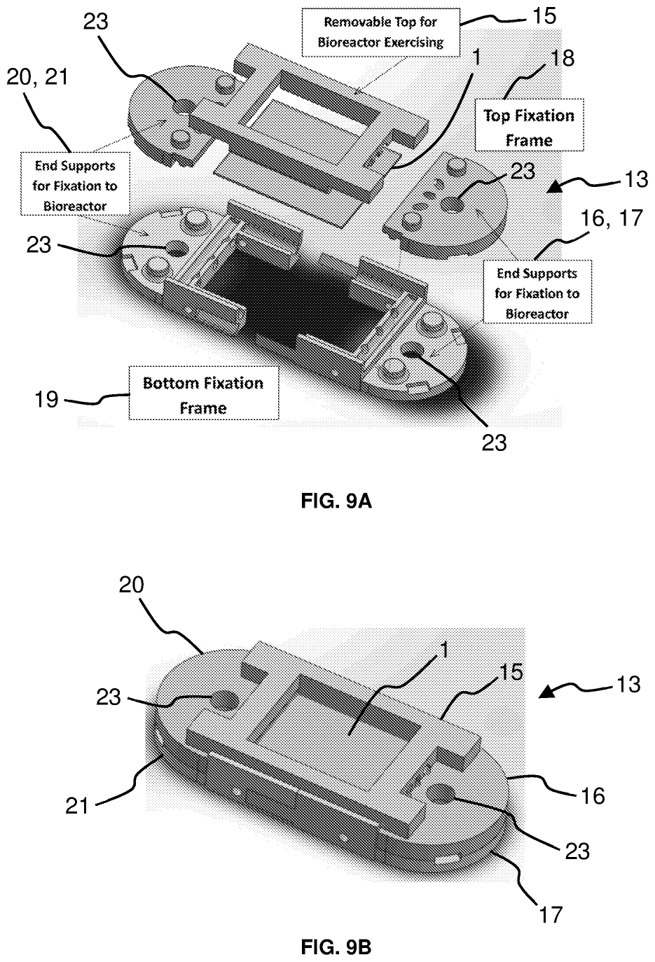

FIGS. 9A and 9B schematically illustrate an exploded view and assembled view, respectively, depicting a prototype for the bioassembly device 13 holding a BAM scaffold 1 and functioning as a seeding chamber. The BAM scaffold 1 is held in place, at least in part, by a top 15 which may be removable. The bioassembly device 13 will enable high resolution cell seeding with a 3D bioprinter 31 (not shown in FIG. 9), prior to insertion into a custom-designed bioreactor 41 (not shown in FIG. 9) or other designated bioreactor. The upper and lower end supports 16, 17 and upper and lower end supports 20, 21 depicted may fit directly into the prongs 55 (not shown in FIG. 9), protrusions, pegs, threaded holding screws or the like of the bioreactor and may be secured in place with nylon bolts, other attachment means, other fastening mechanism, clamps, or the like (not shown in FIG. 9)—allowing cyclic mechanical or static stretch with minimal perturbation of TEMR. The recesses 23 of the bioassembly device 13 may be secured by prongs, protrusions, pegs, or screws on the bioreactor 41 (not shown in FIG. 9) and/or plate 51 (not shown in FIG. 9) that may positioned on a bioprinter during the printing operation. A variety of fastening and attaching mechanisms may be used such as clamps, male-female fittings, peg and hole fittings, sockets, tongue and groove, other fastening mechanisms, other attachment mechanisms, or other means for securing the bioassembly device to the bioreactor. Generally shown are components serving as a top portion such as a top fixation frame 18 and a bottom portion such as a bottom fixation frame 19. The bioassembly device 13 may be provided with a variety of attachment and fastening mechanisms for the purpose of securing the scaffold to the bioassembly device. Some examples may include clamps, clamp-like structures, or presses. The bioassembly device 13 also allows for printing on both sides of the scaffold 1. In other embodiments or approaches, the bioassembly device 13 also enables 3D bioprinting of multiple cell layers, including additional (even multiple) cell types (e.g., endothelial, neuronal, etc.,) with high spatial resolution to mimic desired cellular/tissue stoichiometries and composition required for improved tissue engineered products.

### Bioprinting Process

An aspect of an embodiment of the present invention bioprinting method and system have overcome a broad number of manufacturing challenges. An aspect of an embodiment of the present invention bioprinting method and system provide, but not limited thereto, the following characteristics and advantages s: 1) reproducible deposition of cells/material, 2) automation and reduction of labor, 3) reduction of manufacturing cost/time, 4) method compatibility across cell types, and 5) development of a closed-loop system.

An aspect of an embodiment of the present invention next-generation bioprinted TEMR biofabrication process from bioink formulation to bioreactor preconditioning include a variety of steps and activities, some of which may include, but not limited thereto, the following: 1) choosing a bioink material and developing methods to combine cells homogenously throughout the gel while maintaining viability, 2) developing methods to load the syringe with minimal shear force and introduction of air bubbles, 3) developing a holder to drape the BAM taut and provide a relatively flat surface for printing, 4) developing a reliable method for zeroing the printhead on the ECM scaffold—reducing shear to preserve cell viability, while ensuring an even, precise print, and 5) ensuring that the system allows for bioreactor preconditioning of the cells on the scaffold with future possibility of automation.

In preparation for an aspect of an embodiment of the present invention bioprinting, the BAM scaffold 1, or other type of scaffold as desired or required, is draped over the bioassembly device 13 (See FIG. 3A) having two recesses 23. An aspect of an embodiment of the present invention may include a bioprinting method that may begin with cell harvesting and resuspension in media at a concentration between 3.5×10⁶ and 8.5×10⁶ MPCs/mL which corresponds to 1.4-3.5×10⁵ MPCs/cm² when printed. Hyaluronic acid (HA) is added to the cell suspension to form a 2% HA bioink, which is then loaded into a syringe 35 having a plunger 37, such as a 2.5 mL Hamilton syringe (or other desirable syringe type) with a 500 μm needle (See FIG. 3B). The syringe 35 may be placed in a printhead 33 of a printer 31, such as the Organovo NovoGen® bioprinter. The dissolvable HA bioink is extruded onto the BAM scaffold 1 in a 500 μm thick layer and retains its integrity in the pattern of a filled-in, 21×16 mm rectangle (See FIGS. 3B and 3C). In an embodiment, the bioprinting methods allow 24 hours for the cells to settle and adhere to the BAM scaffold 1, although this will be further optimized (as discussed herein). After 24 hours, the BAM scaffold 1 is flipped over and the opposite side is seeded using the same bioprinting method. Alternatively, not shown, the BAM scaffold 1 may remain in place and the printer is accessible to both sides of the BAM scaffold 1. Further yet, an embodiment may include both the position on the BAM scaffold and the print head changing positions to gain access to any sides or contours of the intended target to achieve specified printing.

In continuation of an aspect of an embodiment of the present invention TEMR biomanufacturing process, the seeded BAMs are transferred to differentiation media in the aforementioned cyclic stretch bioreactor 41 after another 24 hrs (see FIG. 3D). (It is noted that turning to FIG. 8D, illustrated is a photographic depiction of an aspect of an embodiment of the present invention bioassembly device 13 in a bioreactor 41). The hyaluronic acid bioink that is used for TEMR manufacturing is not crosslinked and quickly dissolves in media during the bioreactor preconditioning phase. The optimized manufacturing timeline required to produce a TEMR construct (myoblasts and myotubes) with similar or improved functional regeneration following implantation in vivo, relative to current manufacturing methods, remains to be determined; and is considered part of the present invention, and may be employed within the context of the invention.

### EXAMPLES

Practice of an aspect of an embodiment (or embodiments) of the invention will be still more fully understood from the following examples and experimental results, which are presented herein for illustration only and should not be construed as limiting the invention in any way.

### Example and Experimental Results

### Assessing Cell Coverage and Cell Type Compatibility

In order to assess the reproducibility and heterogeneity of the cell-laden bioink, immortalized mouse myoblasts (C2C12s) were printed onto glass slides. The 2% HA bioink was prepared with C2C12s as described above, and eight rectangular constructs (21 mm×16 mm×0.5 mm) were printed consecutively. Each print consisted of 138 μL of gel, resulting in a total of more than 1.1 mL of gel deposited. After 24 hours in culture, cells were stained using ReadyProbes® for F-actin and DAPI. Confocal microscopy with a 10× objective was used to perform a tile scan of the entire 21 mm×16 mm printed area for each print.

Another set of experiments explored the compatibility of these bioprinting methods with various cell types relevant to skeletal muscle tissue engineering. This included human skeletal muscle progenitor cells, human neurons, mouse endothelial cells, and C2C12s (immortalized mouse myoblasts; see cell sources below). Briefly, each of these cell types were combined into 2% HA gel and printed onto the BAM scaffold either individually, or in co-culture as further described below. Human muscle progenitor cells (hMPCs) were printed alone in 2% HA, then stained for DAPI and F-actin after 24 hours. The hMPCs were also printed in combination with human neurons. For this co-culture, the hMPCs were printed, then the human neurons were printed after 24 hours. These samples were stained for β III tubulin, desmin, and DAPI, and imaged after 13 days. The C2C12s were printed alone and stained for F-actin and DAPI after 24 hours. Finally, the C2C12s were also printed with endothelial cells by combining both cell types into a single bioink. These co-culture samples were stained for CD31, desmin, and DAPI, and imaged 4 days after printing.

### Cell Sources

Human skeletal muscle progenitor cells were obtained by isolation from discarded human samples, using a 2% collagenase digestion, according to established methods. Human neurons were derived from human induced pluripotent stem cells (hiPSCs). The hiPSCs were provided by the University of Virginia Stem Cell Core and differentiated into neurons. The endothelial cells used in these studies were mouse primary bladder endothelial cells obtained from CellBiologics (Chicago, Ill.).

### Results

Overcoming Technical Challenges of Bioprinting on dECM Sheets

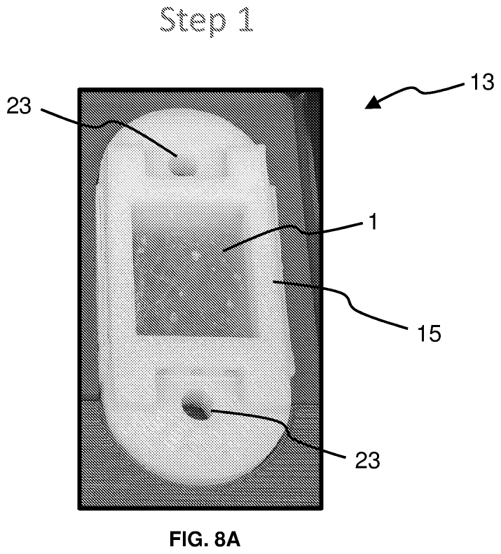

There were at least three key technical challenges of printing on sheet-based scaffolds that would not only enable creation of bioprinted TEMR, but also enable bioprinting on sheet-based scaffolds for diverse research and clinical applications (see FIG. 3, for example, but not limited thereto). A first aspect an embodiment includes developing and configuring a bioassembly device onto which the BAM could be tightly secured (shown in FIG. 3A for example). The design characteristics for this device includes, but not limited thereto: 1) the ability to hold the BAM taut throughout the printing and culturing process, 2) transferability between the printing stage and the bioreactor, with the potential for future automation of these actions, 3) material compatibility with cells in media, and 4) compatibility with the ethylene oxide sterilization for the BAM. An aspect of an embodiment of the resulting device (shown in FIG. 3D for example) was 3D printed with polycarbonate using the Stratasys Fortus 400 printer. A second aspect of an embodiment includes creating a universal stainless steel printing plate 51 (or other material as desired or required) adaptable to the dimensions of a majority of commercially available bioprinters 31 or other type of bioprinter as desired or required. This initial plate design (shown in FIGS. 3B and 6C, for example) allows for the simultaneous printing of three scaffolds at once, and inter-operability between distinct bioprinters—allowing present embodiment method (and related system) to harness the strengths of multiple bioprinting platforms. The plurality of docking locations 53 or area/real estate indicate the accommodation for each of the bioassembly devices 13. Moreover, if the capacity and real estate of the bioprinter 31 or plate 51 were increased and/or the size of the bioassembly 13 decreased then more than three bioassembly devices may be effected/implemented. A third aspect of an embodiment includes determining the proper z-height for effectively printing on a dECM scaffold. In that regard, while the Organovo printer can automatically zero transparent plates using laser optics, the opaque dECM prevents appropriate utilization of this feature. Thus, the z-height for the printer had to be manually determined, which required development of new protocols, as well as implementation of a different format for writing design scripts. In summary, the design solutions to these technical challenges permit a broader range of applications for bioprinting a cell-laden gel onto dECM sheets, or sheets comprised of other relevant biomaterials. Further, a variety of fastening and attaching mechanisms may be used such as clamps, male-female fitting, sockets, peg and hole fittings, or other means for securing the bioassembly to the plate.

Reproducible, Quantifiable Deposition Achieved with Bioprinting Approach

There are two prominent types of extrusion-based bioprinters—printers with pneumatically-driven extrusion and printers with piston-based extrusion, as shown in FIG. 6. One important capability common to all extrusion-based bioprinters is the ability to deposit cells onto a substrate in specific locations in a way that enables patterning of cell populations into configurations that mimic anatomically-relevant architectures. With pneumatic printheads 33, small changes in gel viscosity or pressure settings can greatly affect the amount of gel deposited. Thus, minor inconsistencies in gel viscosity can generate large variations in cell number deposition.

The Organovo printer 31 utilizes piston-driven extrusion printing method, where the plunger 37 of the Hamilton syringe 35 is mechanically depressed in controlled, discrete increments. The rate of extrusion is a programmed parameter, which allows for consistent volumes of deposition every print, regardless of gel viscosity. The volume of gel deposited is measured using the graduations present on the syringes 35. The ability to print discrete, consistent volumes allows for deposition of a specific number of cells. Conversely, the commercially available pneumatically driven printers 31 have advantages that include the ability to print complex CAD files. As previously mentioned, the unique design of an aspect of an embodiment of the present invention bioassembly device 13 and plate 51 allows for interoperability between different types of commercially available bioprinters 31 (shown in FIG. 6), including the 3D-Discovery (RegenHu) illustrated in FIG. 6B and the BioX (CellInk) illustrated in FIG. 6A.

FIGS. 6A-B schematically illustrate two extrusion-based bioprinting methods. The syringe 35 and set of printers 31 are driven pneumatically by using air pressure and the associated syringes lack the graduations necessary for quantifying volumes dispensed. FIG. 6C schematically illustrate a direct mechanical-based bioprinting methods. The Hamilton syringe 35 and Organovo NovoGen bioprinter 31 having a print head 33 is driven by direct mechanical force on the plunger 37. The Hamilton syringe features graduations for exact volume quantification. The plurality of docking locations 53 or area/real estate indicate the accommodation for each of the bioassembly devices 13. Moreover, if the capacity and real estate of the bioprinter 31 or plate 51 were increased and/or the size of the bioassembly 13 decreased then more than three bioassembly devices may be effected/implemented.

### Homogenous Cell Distribution and Print Reproducibility

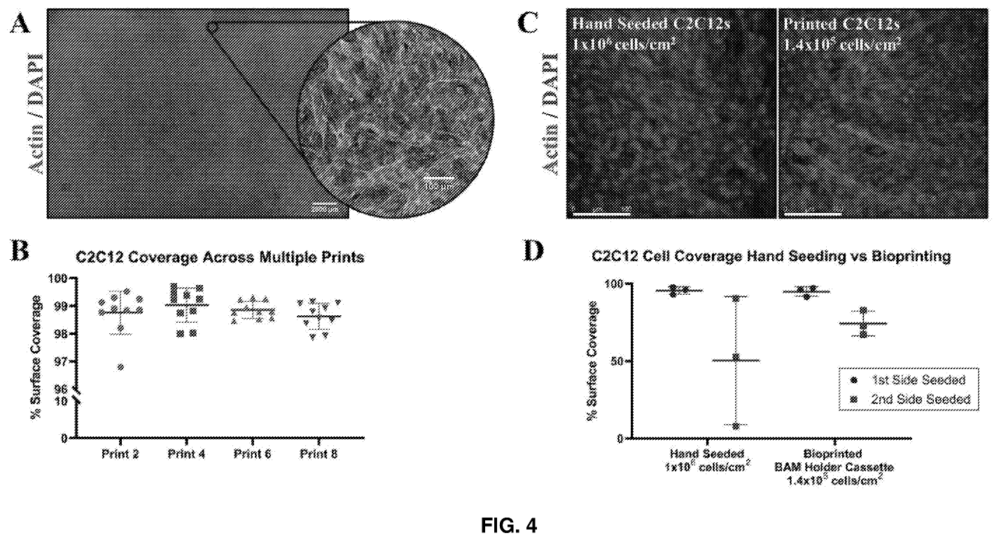

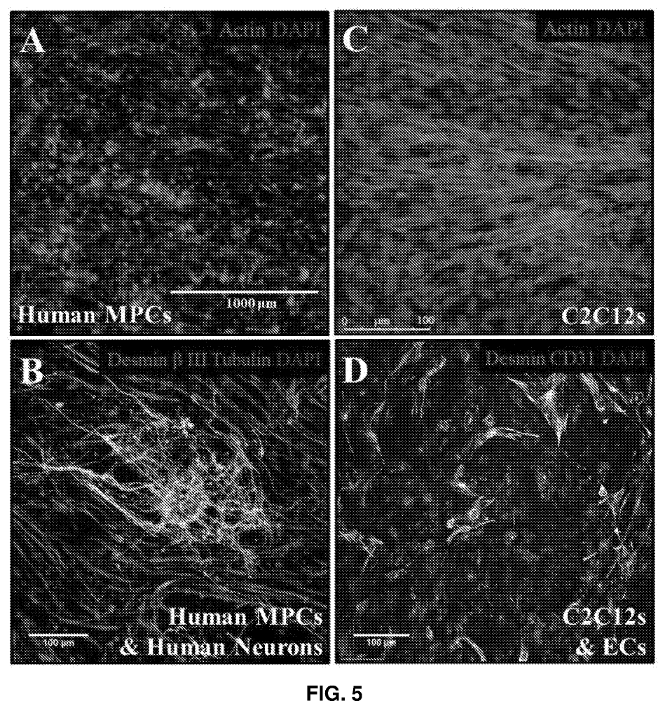

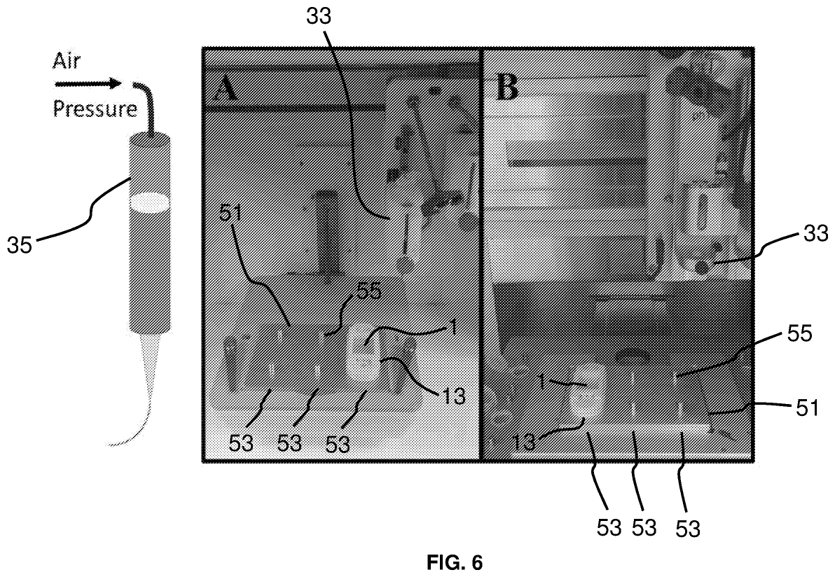

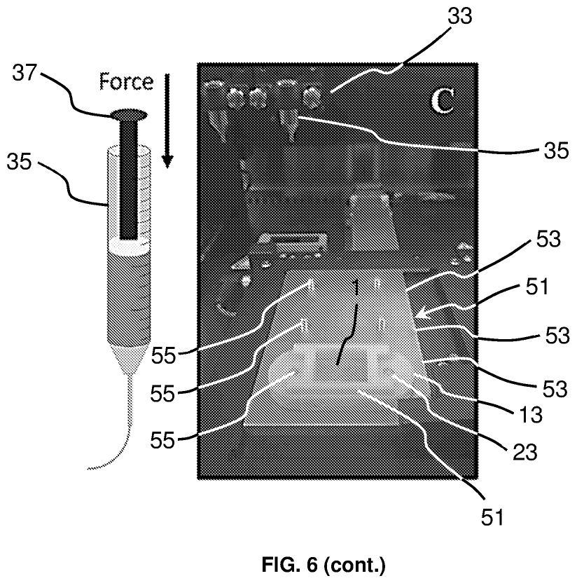

Determining the homogeneity of cell distribution throughout the bioink and the reproducibility of cell homogeneity from construct to construct is an important aspect of an embodiment of the present invention for establishing quality control metrics for the TEMR manufacturing process. Towards this end, homogeneity among eight consecutive prints was assessed and the resulting composite image of a representative print is shown in the micrographic depiction in FIG. 4A. As illustrated, each of the eight composite images had similarly consistent, dense cell coverage.

In this scenario, quantification of the surface area covered by cells was used as an approximation of the relative homogeneity of the prints—both within a single print and across print replicates. Surface coverage of cells on the slides was quantified for 10 images (pre-composite) in four randomly selected representative prints: print #2, 4, 6, and 8 (FIG. 4). Ten random fields of view were selected from locations throughout each entire print then imaged. Surface coverage was quantified in ImageJ by thresholding out the black or near-black pixels which did not contain green (actin) or blue (DAPI) stain, and thus did not have cells present. The percent of the image covered by cells was calculated by dividing the non-black pixels by the total pixels. The results graphically shown in FIG. 4B indicate that the four quantified prints had over 98% cell coverage with extremely low standard deviations (all <1% of the mean). This indicates that cell coverage is reproducible both within prints and between prints. While it was visually clear that each print had similar and consistent cell coverage, this quick quantification confirmed the qualitative observations. Overall, these data are consistent with the supposition that the cells are sufficiently evenly distributed throughout the bioink in a way that permits reproducibility from print-to-print.

FIG. 4 illustrates the reproducible cell coverage at a lower cell density as associated with an aspect of an embodiment of the present invention. Turning to FIG. 4A, provided is a representative composite image of C2C12 cells printed onto a glass slide at a density of 2.9×10⁵ cells/cm². Even cell distribution shown by presence of DAPI (blue) and F-Actin stain (green). The entire 21×16 mm print area shown, scale bar=2000 μm, inset scale bar=100 μm. Turning to FIG. 4B, provided is a quantification of percent coverage from four representative prints. Coverage by cells was quantified in 10 randomly selected images using ImageJ. Standard deviations were all <1% of the mean. Turning to FIG. 4C, provided is a representative images of BAMs that have been manually seeded or bioprinted with C2C12s. The C2C12s were stained with DAPI (blue) and F-actin (red), as shown in the micrographic depiction. Manual seeding requires 5.4×10⁶ cells per side while bioprinting allows for similar coverage at just 7.5×10⁵ cells per side—a 7-fold reduction in the number of cells. Turning to FIG. 4D, provided is preliminary trends of surface coverage of both sides of BAMs seeded with C2C12s using manual seeding and bioprinting methods. One BAM per group was manually seeded at a density of 1×10⁶ cells/cm², and bioprinted in the specially designed cassette at 1.4×10⁵ cells/cm². Cell coverage was quantified by staining the cells with DAPI and F-actin, imaging 3 10× objective FOVs for each side of the BAM, and using ImageJ to threshold and exclude pixels without cells present. For both FIG. 4 and FIG. 4D, percentage of cell coverage was obtained by dividing the non-black pixels (with cells present) by total pixels.

### Reduced Biomanufacturing Time and Cost

The following results from initial proof of concept studies (using C2C12s) demonstrate the feasibility of using bioprinting to: 1) reduce the number of required cells (and thus reduced media and supplies cost); and 2) increase the homogeneity and reproducibility of cell coverage on both sides of the scaffold. Specifically, the current method of manual seeding utilizes 5.4×10⁶ cells per side (1×10⁶ cells/cm²), in large part, to compensate for inefficiencies in the seeding process. Whereas, an aspect of an embodiment of the present invention provides for the bioprinting cells to be encapsulated in a gel that allows for nominally better cell retention on the seeded area.

As shown in the micrographic depiction in FIG. 4C, an aspect of an embodiment of the present invention bioprinting methods allow for a seven-fold reduction in the number of cells required for seeding (7.5×10⁵ per side vs. 5.4×10⁶), while achieving similar cell coverage (95% vs 95.6% quantified in FIG. 4D). In contrast, manual seeding of cells onto the BAM scaffold results in some cell loss (25-75%) when seeding the second side of the BAM scaffold (data not shown). The present inventor compared cell coverage on a BAM seeded by manual methods at a density of 1×10⁶ cells/cm² (in media), to cell coverage on a BAM seeded by an aspect of an embodiment of the present invention bioprinting methods at a density of 1.4×10⁵ cells/cm² (in gel). Side 1 of each BAM was initially seeded at the aforementioned density, and side 2 was seeded at the same density 24 hours later. After another 24 hours, the BAMs were fixed and stained with DAPI and F-actin, and three representative images (similar to those depicted in FIG. 4C) from each side of each BAM were quantified for cell coverage as described above (see FIG. 4 for details). As shown in FIG. 4D, there was little difference in surface coverage between hand seeding (manual) and bioprinting (of an embodiment) for side 1 of the BAM scaffold. Furthermore, consistent with the images shown in FIG. 4C, quantification of cellular coverage revealed that the BAM scaffold seeded by an aspect of an embodiment of the present invention bioprinting exhibited higher surface coverage compared to the BAM which was manually seeded using seven times as many cells per cm². Moreover, an aspect of an embodiment of the present invention bioprinting also allows for more consistent cell coverage across the surface of the BAM scaffold on side two, as demonstrated by the large spread in coverage for the manually seeded BAM scaffold—where remarkable variability was observed in cellular coverage (see FIG. 4D).

### Compatibility Across Multiple Cell Types in Combination

VML injuries result in the loss of vascular and nerve tissue, in addition to the loss of muscle. In order to develop improved biomimetic skeletal muscle constructs for both in vitro and in vivo applications, multiple cell types, including neurons, endothelial cells, vascular smooth muscle cells, and pericytes must eventually be included. As such, another key feature of an aspect of an embodiment of the present invention bioink and bioprinting system (and related method) is its compatibility with multiple relevant cell types. Thus far, the present inventor has successfully bioprinted human skeletal muscle progenitor cells (hMPCs), human induced pluripotent stem cell (hiPSC)-derived neurons, mouse myoblasts, and mouse endothelial cells (human skeletal muscle progenitor cells (hMPCs), human induced pluripotent stem cell (hiPSC)-derived neurons, mouse myoblasts, and mouse endothelial cells (ECs)) onto the BAM scaffold, using the aforementioned 2% HA bioink.

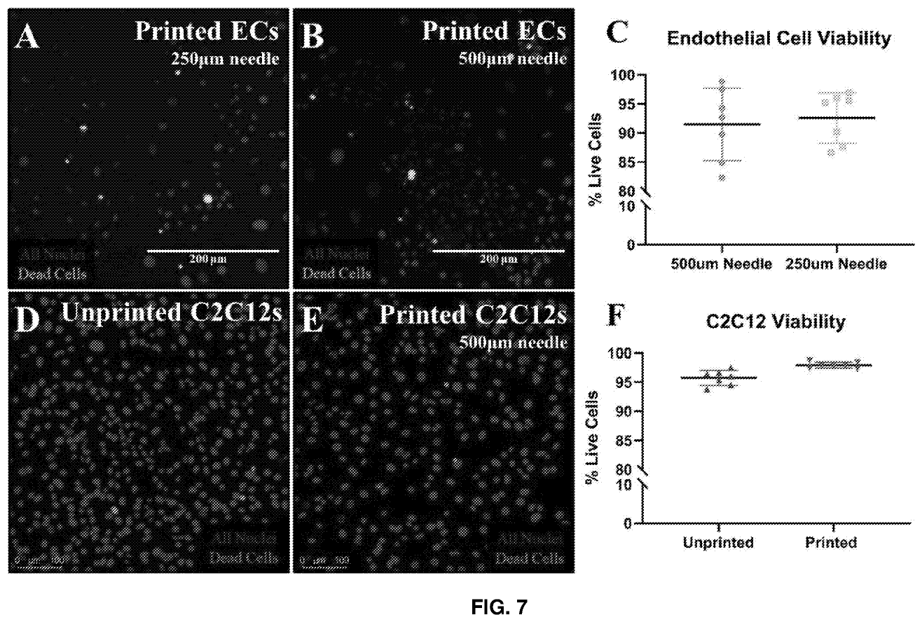

FIG. 5A is a micrographic depiction that shows hMPCs printed alone at a density of 1.85×10⁵ hMPCs/cm². The co-culture of hMPCs and human neurons shown in the micrographic depiction in FIG. 5B consisted of human muscle progenitor cells printed first at Day 0 at a density of 3.7×10⁵ hMPCs/cm². After 24 hours, the human neurons were printed at a density of 3×10⁴ neurons/cm². These samples were imaged after 13 days in culture. Importantly, the human neurons printed in co-culture with human MPCs depicted in the micrographic depiction in FIG. 5B are shown to extend branched dendrites, indicating healthy neuron activity and potentially functional interaction with muscle cells. The C2C12s in both FIGS. 5C and 5D (provided in their micrographic depictions) were printed at a density of 1.8×10⁵ cells/cm². In FIG. 5D, the C2C12s were printed in direct combination with the mouse bladder endothelial cells at a density of 2.4×10⁵ ECs/cm². These samples were imaged after 4 days in culture (see micrographic depiction as shown in FIG. 5D). Taken together, the range of cell types printed thus far and the ability to co-culture these cell types suggests that an aspect of an embodiment of our present invention bioprinting methods (and related systems) are beneficial to not only automating the MPC seeding process, but also for incorporating and patterning multiple relevant cell types in the TEMR construct.

As discussed above, FIG. 5 demonstrates, in part, an initial application of dECM bioprinting to other relevant cell types. FIG. 5A includes 1.85×10⁵ hMPCs/cm² stained with DAPI (blue) and F-Actin (red) after 24 hrs, wherein scale bar=1000 μm. FIG. 5B includes 3.7×10⁵ hMPCs/cm² printed at t=0 and 3×10⁴ human neurons/cm² printed at t=24 hrs. Scaffolds were stained after 13 days with DAPI (blue), desmin (MPCs, red), and β III tubulin (neurons, green), wherein scale bar=100 μm. FIG. 5C includes 1.8×10⁵ C2C12s/cm² stained with DAPI (blue) and F-Actin (red) after 24 hrs, wherein scale bar=100 μm. FIG. 5D includes 1.8×10⁵ C2C12s/cm² printed together with 2.4×10⁵ ECs/cm² stained with desmin (C2C12s, pink), CD31 (ECs, green), and DAPI (blue), and imaged after 4 days in culture, wherein scale bar=100 μm.

Bioprinting with High (>90%) Cell Viability

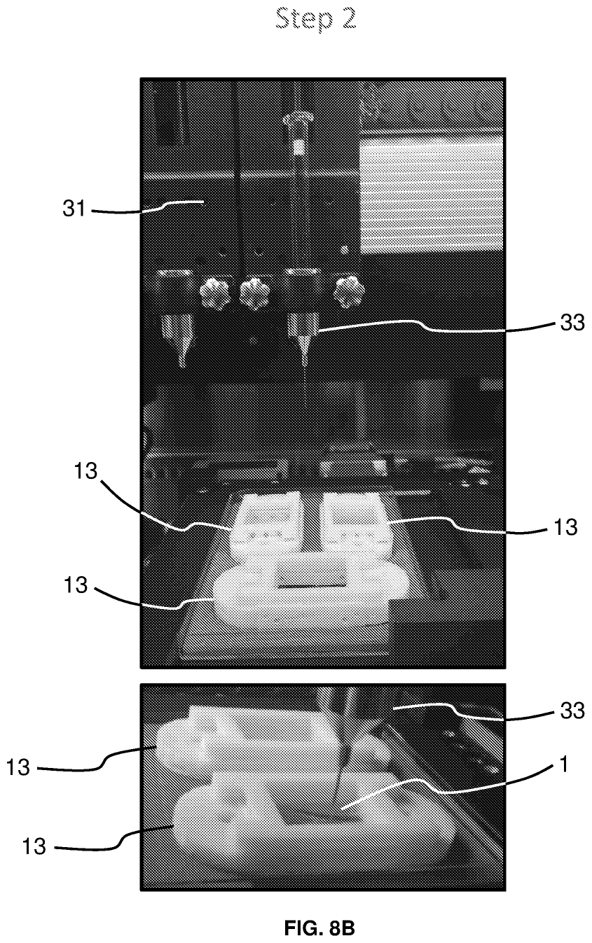

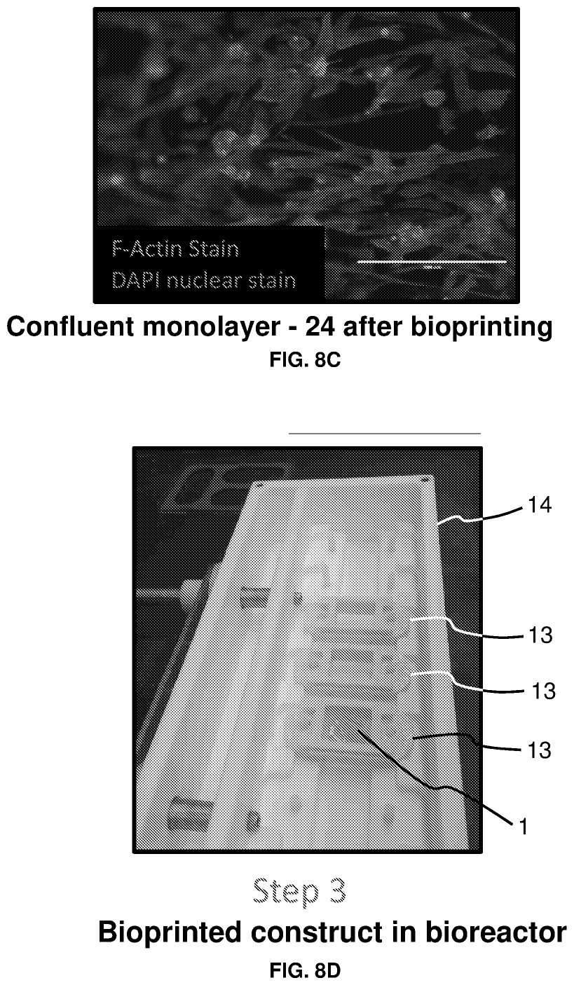

The viability of several additional cell types was initially assessed for the Organovo NovoGen 3D bioprinter. The printer settings used were a lateral speed of 5 mm/s, an extrusion rate of 25-50 μm/s, and a z displacement of 250-500 μm between the printing surface and the needle of the Hamilton syringe 24 hours after extrusion. As shown in FIG. 7, these preliminary results indicate that at these settings high cell viabilities (>90%) were achieved for both mouse myoblasts and endothelial cells using either a 250 μm or 500 μm needle diameter.

Referring to FIG. 7, cell viability 24 hours after bioprinting is presented. Turning to FIG. 7A, primary endothelial cells (CellBiologics cat #C57-6214 at passage 11) printed in 2% HA gel through a 250 μm needle and, and as shown in FIG. 7B, through a 500 μm needle onto a 6-well cell culture dish. After 24 hours, all cells were stained with DAPI (blue) and dead cells were stained green (ReadyProbes® Cell Viability Imaging kit), scale bar=200 μm. Turning to FIG. 7C, seven random representative 10× objective images were taken from one printed area per group and both all cells and all dead cells were counted. From these counts, the percent of live cells was calculated. Turning to FIG. 7D-7E C2C12s were encapsulated in 2% HA gel at a concentration of 4.7×10⁶/mL and dispensed onto glass slides by: as shown in FIG. 7D hand seeding and as shown in FIG. 7E, printing through a 500 μm needle. After 24 hours, all cells stained with DAPI (blue) and dead cells were stained red, wherein scale bar=100 μm. Turning to FIG. 7F, percent live cells quantified as above in FIG. 7C.

### Discussion

### Manufacturing and Technical Advantages of Novel Biofabrication System

Overall, the various aspects of embodiments of the TEMR process described herein presents potentially important solutions to several biomanufacturing challenges such as automation, reproducibility, and time and cost reduction (cost of goods; COGs). These advantages are highlighted in Table 1. Although further rigorous investigations with more clinically relevant cells (e.g., human myoblasts) are required for confirmation of these findings with C2C12 cells, these initial observations demonstrate the presence of a reproducible and established cell monolayer 24 hours following bioprinting. The implication is that minimally, an aspect of an embodiment of the present invention shall provide for the ability to create uniform and homogeneous cell populations on both sides of the scaffold with a ≈7-fold reduction in the number of cells required. This may also reduce the manufacturing time line prior to bioreactor preconditioning—in effect resulting in a potential 30-85% reduction in the overall timeline for TEMR production.

As previously mentioned, an aspect of an embodiment of the present invention provides for the bioassembly device and printing plate that lends itself to, among other things, a more automated, and eventually, closed-loop system. This early stage proof of concept work lays the basis for the further development of a fully-automated, closed loop system from cell seeding to TEMR construct completion. This would be a system in which the cells could be printed on the BAMs, and then cultured, differentiated, and preconditioned all within the same bioreactor device. This approach would further reduce the manual labor required for biofabrication of TEMR, and thus, accordingly reduce the cost associated with production. When considering the biofabrication process for TEMRs in a good manufacturing practices (GMP) facility, a fully-automated, closed-loop system would also be beneficial for maintaining sterility of the product and minimizing contamination.

TABLE 1
Technical Advantages of an Aspect of an embodiment
of the Biofabrication System and Related Method

Reduced Manual Operations: Automated rather than manual cell seeding
Increased ease of use bioreactor placement: modular chamber rather than
individual scaffold placement/manipulation (especially in the presence of
cells)
Reduced timeline: 1 day to uniform confluence on both sides of the
scaffold rather than 10 days (90% reduction)
Reduced # of cells required: 150-350,000 cells/cm² rather than 1,000,000
cells/cm² (3-10-Fold reduction)
Improved controller interface with bioreactor: to enable monitoring of
motion, temperature, metabolites

### Application Advantages of Novel Biofabrication System Beyond Skeletal Muscle

The biomanufacturing methods described are somewhat analogous to cell sheet technologies—another area of biofabrication research that yields cell-dense constructs. However, an aspect of an embodiment the present invention manufacturing methods for bioprinting TEMR differ from cell sheets in that an aspect of an embodiment of the bioprinting offers controllable deposition of both cell types and cell numbers, and the supporting dECM substrate itself plays a critical role in the construct. This robust, but ultimately biodegradable dECM allows force transduction to the differentiating muscle progenitor cells, facilitating cellular organization and unidirectional orientation during cyclic mechanical stretch preconditioning in the bioreactor. The dECM material is also suturable, and thus ideal for surgical implantation, ultimately enabling an improved interface with surrounding native tissue. Eventually, the dECM scaffold will degrade, leaving only remodelled/repaired/regenerated tissue structure(s) behind.

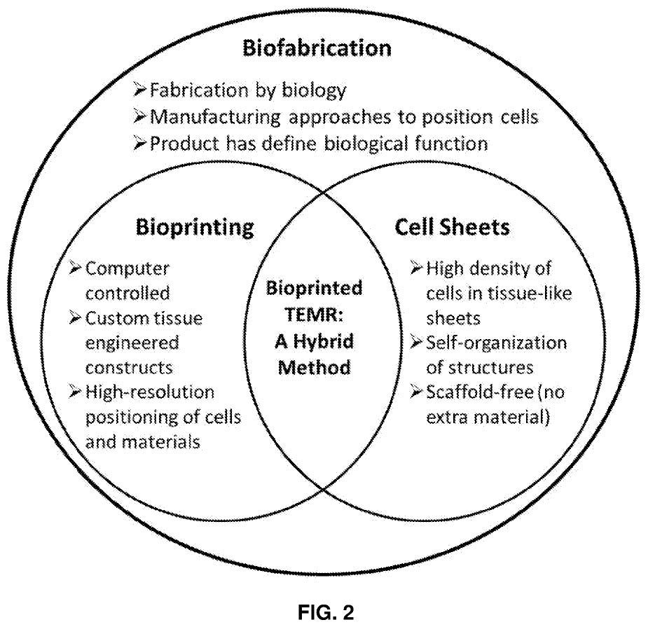

This hybrid approach of using bioprinting to establish cell sheets supported by a degradable substrate thus leverages strengths of both computer-directed printing and self-assembly (for example, see FIG. 2). In particular, the hybrid approach allows for, among other things, homogeneity in cellular coverage on both sides of the BAM scaffold, as well as a dramatic reduction in the number of cells required to achieve improved cell seeding density and consistency. Bioprinting also ameliorates many biomanufacturing challenges and offers the ability to fabricate a construct in a way that might be streamlined towards an industrial-inspired biomanufacturing-type process.

Still referring to an aspect of an embodiment of the present invention, beyond the technical advantages that should result in accelerated biomanufacturing and reduced costs, the sheet-based platform has many potential application advantages as well (for example, but not limited thereto, see Table 2). Overall, there is considerable flexibility in a sheet-based tissue engineering platform to produce implantable constructs with very distinct geometries. Specifically, the rationale for the initial application of an embodiment of the present invention construct for craniofacial reconstruction, is related to the sheet-like nature of many of the facial muscles, for example, the orbicularis oris muscle of the lip that is the locus of cleft lip deformities. In addition, an aspect of an embodiment of the present invention system is able to leverage the double-sided printing capabilities, which has important implications for extending the range of applications. For example, tissues such as blood vessels and gastrointestinal tract could be created by printing endothelial or epithelial cells, respectively, on one side of the scaffold and smooth muscle cells on the other—followed by rolling the construct into a tubular shape. Various bioengineered constructs could leverage an aspect of an embodiment of the present invention bioprinting system described herein, and yet serve to provide tissue constructs for distinct replacement/reconstruction purposes.

In addition, an aspect of an embodiment of these sheet-like constructs of the present invention can be folded in unique ways to produce a sac-like (bag) structure that might be amenable, for example, to bladder reconstruction. There is also no obvious constraint on the size of the constructs that can be seeded, so there is opportunity for significant scalability to meet the needs of larger reconstructive procedures. In an embodiment, the constructs could also be stacked in vivo, over time, to produce even larger volumes of tissue reconstitution. This is consistent with the present inventor's published¹¹⁻¹³ and unpublished data where implantation of TEMR constructs that range from ≈500 μm to −1 mm in thickness results in robust volume reconstitution of several millimeters in tissue thickness. While engineered constructs are often limited by the diffusion distance of oxygen, TEMR has been shown to have therapeutic effects after implantation without the presence of mature vasculature—as documented by the preclinical success of TEMR implantation^{8-15,26}. This is presumably related to the fact that following TEMR implantation, vasculature is able to infiltrate the construct without requiring a mature vasculature in the construct itself, at the time of implantation. Finally, an aspect of an embodiment of the present invention multiple cell types can eventually be added (bioprinted) to the constructs with high spatial resolution, which when combined with additional bioreactor incubation and conditioning protocols, can produce more mature constructs, with diverse applications for biological assays (in vitro) and clinical implants (in vivo). Again, all of these advantages are summarized, at least in part, in Table 2.

TABLE 2
Application Advantages of an Aspect of an Embodiment
of the Biofabrication System and Related Methods

Flexible geometry
Folding: Bags (e.g., bladder)
Rolling: Tubes (e.g., blood vessels, intestinal tract, ureter, urethra, etc.)
Stacking of individual constructs: enhanced in vivo volume reconstitution
Scalability: much larger constructs can be made, because the nutrient
requirements of confluent scaffolds is achievable at many size scales
Multiple cell type applications: Additional cell types can be added with
high spatial resolution of Organovo bioprinter and ease of placement
coordinates on sheet-like scaffold.
Minimal nutrient requirements also mean that sheet-like scaffolds (i.e.,
2-3 mm) thickness at implantation and can be easily integrated
(vascularized) into host tissue.

### Non-Limiting Conclusions

Overall, an aspect of an embodiment of the present invention provides a bioprinting approach, method and system that, among other things, employs bioprinting in a non-classical method, which allows for printing high densities of cells onto sheet-like scaffolds. An aspect of an embodiment of the present invention provides an important step forward with respect to addressing very important technology gaps for the field. Moreover, an aspect of an embodiment of the present invention system and method have the potential to significantly reduce biofabrication time lines and manufacturing costs, while maintaining an open design architecture to ensure a seamless transition for any future biomanufacturing requirements. The preliminary results discussed and disclosed herein document the initial feasibility of using an aspect of an embodiment of the present invention bioprinting methods and systems to reduce the time and cost associated with biofabricating tissue engineered constructs.

In the current instance, the TEMR construct was highlighted as an example of the potential utility of this technology. Using an aspect of an embodiment of the present invention provides for bioprinting as part of the TEMR biofabrication process should enable creation of more uniform and homogeneous cell populations on both sides of the scaffold with a ≈7-fold reduction in the number of cells required. This may eventually also reduce the manufacturing time line prior to bioreactor preconditioning by as much as 90%. Certainly, further characterization and optimization may be required and is considered part of the present invention, and may be employed within the context of the invention. Nonetheless, the increased efficiencies, diminished production timelines and costs, and the wide range of potential clinical applications bode well for the utility of an aspect of an embodiment of the present invention approach, method and system as an attractive biomanufacturing platform—with promise for accelerating the application of tissue engineering/regenerative medicine technologies for diverse unmet clinical needs.

### Additional Examples

### Example 1

An aspect of an embodiment of the present invention provides, among other things, a bioprinting method, wherein the method may comprise: disposing a scaffold onto a bioassembly device; disposing the bioassembly device, with the scaffold, onto a bioprinter; bioprinting onto a first side of the scaffold or both the first side and a second side of the scaffold, which is disposed on the bioassembly device that is disposed on the bioprinter; transferring the bioprinted scaffold, which is disposed on the bioassembly device, onto a bioreactor; and creating tissue engineered construct while the bioprinted scaffold remains on the bioassembly device and in the bioreactor.

### Example 2

The method of example 1, wherein the scaffold comprises a sheet-based scaffold.

### Example 3

The method of example 1 (as well as subject matter in whole or in part of example 2), wherein the tissue engineered construct comprises at least one or more of any combination of the following:

implantable tissue engineered construct;

three dimensional structure tissue engineered construct;

solid organs construct;

organoids construct;

sheet-like construct;

varying geometrical shapes of the construct; and

distinct consistency on a first side of the contrast relative to a second side of the construct.

### Example 4

The method of example 3 (as well as subject matter in whole or in part of example 2), further comprising:

folding the sheet-like construct.

### Example 5

The method of example 3 (as well as subject matter of one or more of any combination of examples 2 or 4, in whole or in part), further comprising:

repeating steps of example 1 one or more times, and stacking two or more of the constructs.

### Example 6

The method of example 1 (as well as subject matter of one or more of any combination of examples 2-5, in whole or in part), wherein the bioprinting includes directly depositing cells onto the first side of the scaffold or both the first side and a second side of the scaffold.

### Example 7

The method of example 6 (as well as subject matter of one or more of any combination of examples 2-5, in whole or in part), wherein the bioprinting comprises encapsulating the cells being depositing in a gel.

### Example 8

The method of example 6 (as well as subject matter of one or more of any combination of examples 2-5 and 7, in whole or in part), wherein the bioprinting comprises controlling the number of cells being deposited and/or type of cells being deposited.

### Example 9

The method of example 1 (as well as subject matter of one or more of any combination of examples 2-8, in whole or in part), wherein the bioprinting includes extruding bioink onto the first side of the scaffold or both the first side and a second side of the scaffold.

### Example 10

The method of example 9 (as well as subject matter of one or more of any combination of examples 2-8, in whole or in part), wherein the bioink comprises at least one or more of any combination of the following: hyaluronic acid (HA), gelatin, alginate, fibrinogen, collagen, and other biopolymers.

### Example 11

The method of example 1 (as well as subject matter of one or more of any combination of examples 2-10, in whole or in part), wherein the creating comprises: culturing, differentiating, and preconditioning the scaffold in the bioreactor while the scaffold remains on the bioassembly device.

### Example 12

The method of example 1 (as well as subject matter of one or more of any combination of examples 2-11, in whole or in part), wherein the creating comprises:

incubating the bioprinted scaffold.

### Example 13

The method of example 11 (as well as subject matter of one or more of any combination of examples 2-10 and 12, in whole or in part), wherein the creating comprises:

stretching the bioprinted scaffold.

### Example 14

The method of example 1 (as well as subject matter of one or more of any combination of examples 2-13, in whole or in part), wherein the creating comprises:

seeding the first side of the bioprinted scaffold or both the first side and a second side of the bioprinted scaffold.

### Example 15

The method of example 14 (as well as subject matter of one or more of any combination of examples 2-13, in whole or in part), wherein the seeding includes controlling cell seeding density and/or cell seeding consistency.

### Example 16

The method of example 1 (as well as subject matter of one or more of any combination of examples 2-15, in whole or in part), wherein the disposing the scaffold onto the bioassembly device includes securing the scaffold in position for the bioprinting.

### Example 17

The method of example 1 (as well as subject matter of one or more of any combination of examples 2-16, in whole or in part), wherein the disposing the scaffold onto the bioassembly device includes securing the scaffold in a taut position for the bioprinting.

### Example 18

The method of example 17 (as well as subject matter of one or more of any combination of examples 2-16, in whole or in part), wherein disposing the bioassembly device includes securing the bioassembly device to the bioprinter.

### Example 19

The method of example 18 (as well as subject matter of one or more of any combination of examples 2-16, in whole or in part), wherein the securing the bioassembly device to the bioprinter comprises disposing a plate on the bioprinter configured to receive the bioassembly device.

### Example 20

The method of example 18 (as well as subject matter of one or more of any combination of examples 2-17 and 19, in whole or in part), wherein after transferring the bioprinted scaffold that is disposed on the bioassembly device, securing the bioassembly device to the bioreactor.

### Example 21

The method of example 20 (as well as subject matter of one or more of any combination of examples 2-19, in whole or in part), wherein the disposing the scaffold onto the bioassembly device includes securing the scaffold in a taut position while in the bioreactor.

Example 22 An aspect of an embodiment of the present invention provides, among other things, a bioassembly device for use with a bioprinter, wherein the device may comprise: a top portion and a bottom portion that are configured to secure a scaffold there between while the bioprinter performs bioprinting onto a first side of the scaffold or both the first side and a second side of the scaffold.

### Example 23

The device of example 22, wherein the top portion and the bottom portion are configured to secure the bioprinted scaffold while it is transferred to a bioreactor.

### Example 24

The device of example 22 (as well as subject matter in whole or in part of example 23), wherein the top portion and the bottom portion are configured to:

slidably connect together with one another; or snap-fit connect with one another one another.

### Example 25

The device of example 23 (as well as subject matter in whole or in part of example 24), wherein the top portion and the bottom portion are configured to secure the transferred bioprinted scaffold in the bioreactor while the scaffold is created into tissue engineered construct.

### Example 26

The device of example 25 (as well as subject matter of one or more of any combination of examples 23-24, in whole or in part) provided in a kit, wherein the kit includes the scaffold.

### Example 27

The device of example 26 (as well as subject matter of one or more of any combination of examples 23-25, in whole or in part), wherein the kit provides the scaffold as the tissue engineered construct that comprises at least one or more of any combination of the following:

implantable tissue engineered construct;

three-dimensional structure tissue engineered construct;

solid organs construct;

organoids construct;

sheet-like construct;

varying geometrical shapes of the construct; and

distinct consistency on a first side of the contrast relative to a second side of the construct.

### Example 28

The device of example 26 (as well as subject matter of one or more of any combination of examples 23-25 and 27, in whole or in part), wherein the kit provides the scaffold in a folded configuration construct.

### Example 29

The device of example 26 (as well as subject matter of one or more of any combination of examples 23-25 and 27-28, in whole or in part), wherein the kit provides two or more the scaffolds wherein the two or more the scaffolds are stacked to form the construct.

### Example 30

The device of example 22 (as well as subject matter of one or more of any combination of examples 23-29, in whole or in part), wherein the top portion and the bottom portion are configured to secure the scaffold there between while cells are deposited onto the first side of the scaffold or both the first side and a second side of the scaffold during the bioprinting.

### Example 31

The device of example 30 (as well as subject matter of one or more of any combination of examples 23-29, in whole or in part), wherein the top portion and the bottom portion are configured to secure the scaffold there between while the cells are encapsulated in a gel during bioprinting.

### Example 32

The device of example 22 (as well as subject matter of one or more of any combination of examples 23-31, in whole or in part), wherein the top portion and the bottom portion that are configured to secure the scaffold comprises at least one or more of the following:

a frame configured to provide the scaffold securement;

a portion of a frame configured to provide the scaffold securement;

a clamp configured to provide the scaffold securement; or

bars or elongated members arranged to provide the scaffold securement.

### Example 33

The device of example 22 (as well as subject matter of one or more of any combination of examples 23-32, in whole or in part), wherein the securing the scaffold while in the bioprinter includes securing the scaffold in a taut position for the bioprinting.

### Example 34

The device of example 22 (as well as subject matter of one or more of any combination of examples 23-33, in whole or in part), wherein the top portion and the bottom portion are configured to be secured in place at a designated location in the bioprinter.

### Example 35

The device of example 23 (as well as subject matter of one or more of any combination of examples 24-34, in whole or in part), wherein the top portion and bottom portion are configured to be secured in place at a designated location in the bioreactor transferred therein.

### Example 36

The device of example 23 (as well as subject matter of one or more of any combination of examples 24-35 in whole or in part), wherein:

the securing the scaffold while in the bioprinter includes securing the scaffold in a taut position for the bioprinting; and

the securing the scaffold while in the bioreactor includes securing the scaffold in a taut position while in the bioreactor.

### Example 37

The device of example 22 (as well as subject matter of one or more of any combination of examples 23-36, in whole or in part) provided in a kit, wherein the kit includes the bioprinter.

### Example 38

The device of example 23 (as well as subject matter of one or more of any combination of examples 23-37, in whole or in part) provided in a kit, wherein the kit includes the bioprinter and the bioreactor.

### Example 39

An aspect of an embodiment of the present invention provides, among other things, a bioprinting system, where the system may comprise: a designated area configured for receiving a bioassembly device, which includes a scaffold disposed in the bioassembly device; and a print head configured for bioprinting onto a first side of the scaffold or both the first side and a second side of the scaffold, while the bioassembly device is in the designated area of the bioprinting system.

### Example 40

The system of example 39, wherein the bioprinting includes directly depositing cells onto the first side of the scaffold or both the first side and a second side of the scaffold.

### Example 41

The system of example 40, wherein the bioprinting comprises encapsulating the cells being depositing in a gel.

### Example 42

The system of example 40 (as well as subject matter in whole or in part of example 41), wherein the bioprinting comprises controlling the number of cells being deposited and/or type of cells being deposited.

### Example 43

The system of example 39 (as well as subject matter of one or more of any combination of examples 40-42, in whole or in part), wherein the bioprinting includes extruding bioink onto the first side of the scaffold or both the first side and a second side of the scaffold.

### Example 44

The system of example 39 (as well as subject matter of one or more of any combination of examples 40-43, in whole or in part), wherein the designated area is configured to secure the bioassembly device to the bioprinting system.

### Example 45

The system of example 39 (as well as subject matter of one or more of any combination of examples 40-44, in whole or in part), further comprising a kit, wherein the system may be provided with a bioreactor, and wherein the bioassembly device is configured to secure the bioprinted scaffold while it is transferred to the bioreactor.

### Example 46

The system of example 45 (as well as subject matter of one or more of any combination of examples 40-44, in whole or in part), further comprising a kit, wherein the system may be provided with a bioreactor, and wherein the bioassembly device is configured to secure the bioprinted scaffold at a designated location in the bioreactor transferred therein.

### Example 47

The method of using any of the devices and systems or their components or sub-components provided in any one or more of examples 22-46, in whole or in part.

### Example 48

The method of manufacturing any of the devices and systems or their components or sub-components provided in any one or more of examples 22-46, in whole or in part.

### Example 49

A non-transitory machine readable medium including instructions for bioprinting, which when executed by a machine, causes the machine to perform any of the steps or activities provided in any one or more of examples 1-21.

### Example 50

A non-transitory computer readable medium including program instructions for bioprinting, wherein execution of the program instructions by one or more processors of a computer system causes the processor to carry out: any of the steps or activities provided in any one or more of examples 1-21.

### REFERENCES

The following patents, applications and publications as listed below and throughout this document are hereby incorporated by reference in their entirety herein, and which are not admitted to be prior art with respect to the present invention by inclusion in this section.
1. Corona B T, Rivera J C, Owens J G, Wenke J C, Rathbone C R. Volumetric muscle loss leads to permanent disability following extremity trauma. Journal of Rehabilitation Research and Development. 2015; 52(7). doi:10.1682/JRRD.2014.07.0165
2. Grogan B F, Hsu J R. Volumetric muscle loss. The Journal of the American Academy of Orthopaedic Surgeons. 2011; 19 Suppl 1:S35-7.
3. Lawson R, Levin L S. Principles of Free Tissue Transfer in Orthopaedic Practice. Journal of the American Academy of Orthopaedic Surgeons. 2007; 15(5):290-9.
4. Norris B L, Kellam J F. Soft-Tissue Injuries Associated With High-Energy Extremity Trauma: Principles of Management. JAAOS—Journal of the American Academy of Orthopaedic Surgeons. 1997; 5(1).
5. Han N, Yabroudi M A, Stearns-Reider K, Helkowski W, Sicari B M, Rubin J P, Badylak S F, Boninger M L, Ambrosio F. Electrodiagnostic Evaluation of Individuals Implanted With Extracellular Matrix for the Treatment of Volumetric Muscle Injury: Case Series. Physical therapy. 2016; 96(4):540-549. doi:10.2522/ptj.20150133
6. Mase V J J, Hsu J R, Wolf S E, Wenke J C, Baer D G, Owens J, Badylak S F, Walters T J. Clinical application of an acellular biologic scaffold for surgical repair of a large, traumatic quadriceps femoris muscle defect. Orthopedics. 2010; 33(7):511. doi:10.3928/01477447-20100526-24
7. Sicari B M, Rubin J P, Dearth C L, Wolf M T, Ambrosio F, Boninger M, Turner N J, Weber D J, Simpson T W, Wyse A, et al. An acellular biologic scaffold promotes skeletal muscle formation in mice and humans with volumetric muscle loss. Science translational medicine. 2014; 6(234):234ra58. doi:10.1126/scitranslmed.3008085
8. Baker H B, Passipieri J A, Siriwardane M, Ellenburg M D, Vadhavkar M, Bergman C R, Saul J M, Tomblyn S, Burnett L, Christ G J. Cell and Growth Factor-Loaded Keratin Hydrogels for Treatment of Volumetric Muscle Loss in a Mouse Model. Tissue Engineering Part A. 2017; 23(11-12):572-584. doi:10.1089/ten.tea.2016.0457
9. Passipieri J A, Baker H B, Siriwardane M, Ellenburg M D, Vadhavkar M, Saul J M, Tomblyn S, Burnett L, Christ G J. Keratin Hydrogel Enhances In Vivo Skeletal Muscle Function in a Rat Model of Volumetric Muscle Loss. Tissue Engineering Part A. 2017; 23(11-12):556-571. doi:10.1089/ten.tea.2016.0458
10. Passipieri J A, Christ G J. The Potential of Combination Therapeutics for More Complete Repair of Volumetric Muscle Loss Injuries: The Role of Exogenous Growth Factors and/or Progenitor Cells in Implantable Skeletal Muscle Tissue Engineering Technologies. Cells Tissues Organs. 2016; 202(3-4):202-213. doi:10.1159/000447323
11. Machingal M A, Corona B T, Walters T J, Kesireddy V, Koval C N, Dannahower A, Zhao W, Yoo J J, Christ G J. A Tissue-Engineered Muscle Repair Construct for Functional Restoration of an Irrecoverable Muscle Injury in a Murine Model. Tissue Engineering Part A. 2011; 17(17-18):2291-2303. doi:10.1089/ten.tea.2010.0682
12. Corona B T, Machingal M A, Criswell T, Vadhavkar M, Dannahower A C, Bergman C, Zhao W, Christ G J. Further Development of a Tissue Engineered Muscle Repair Construct In Vitro for Enhanced Functional Recovery Following Implantation In Vivo in a Murine Model of Volumetric Muscle Loss Injury. Tissue Engineering Part A. 2012; 18(11-12):1213-1228. doi:10.1089/ten.tea.2011.0614
13. Corona B T, Ward C L, Baker H B, Walters T J, Christ G J. Implantation of In Vitro Tissue Engineered Muscle Repair Constructs and Bladder Acellular Matrices Partially Restore In Vivo Skeletal Muscle Function in a Rat Model of Volumetric Muscle Loss Injury. Tissue Engineering Part A. 2013; 20(3-4):705-715. doi:10.1089/ten.tea.2012.0761
14. Christ G J, Passipieri J A, Treasure T E, Freeman P N, Wong M E, Martin N R W, Player D, Lewis M P. Chapter 43—Skeletal Muscle Tissue Engineering. In: Vishwakarma A, Sharpe P, Shi S, Ramalingam MBT-SCB and TE in DS, editors. Boston: Academic Press; 2015. p. 567-592. doi:https://doi.org/10.1016/B978-0-12-397157-9.00047-3
15. Christ G J, Siriwardane M L, de Coppi P. Engineering muscle tissue for the fetus: getting ready for a strong life. Frontiers in pharmacology. 2015; 6:53. doi:10.3389/fphar.2015.00053
16. Valentin J E, Turner N J, Gilbert T W, Badylak S F. Functional skeletal muscle formation with a biologic scaffold. Biomaterials. 2010; 31(29):7475-7484. doi:https://doi.org/10.1016/j.biomaterials.2010.06.039
17. Dziki J L, Sicari B M, Wolf M T, Cramer M C, Badylak S F. Immunomodulation and Mobilization of Progenitor Cells by Extracellular Matrix Bioscaffolds for Volumetric Muscle Loss Treatment. Tissue Engineering Part A. 2016; 22(19-20):1129-1139. doi:10.1089/ten.tea.2016.0340
18. Wolf M T, Daly K A, Reing J E, Badylak S F. Biologic scaffold composed of skeletal muscle extracellular matrix. Biomaterials. 2012; 33(10):2916-2925. doi:10.1016/j.biomaterials.0.2011.12.055
19. Sicari B M, Agrawal V, Siu B F, Medberry C J, Dearth C L, Turner N J, Badylak S F. A murine model of volumetric muscle loss and a regenerative medicine approach for tissue replacement. Tissue engineering. Part A. 2012; 18(19-20):1941-1948. doi:10.1089/ten.TEA.2012.0475
20. Merritt E K, Hammers D W, Tierney M, Suggs L J, Walters T J, Farrar R P. Functional Assessment of Skeletal Muscle Regeneration Utilizing Homologous Extracellular Matrix as Scaffolding. Tissue Engineering Part A. 2009; 16(4):1395-1405. doi:10.1089/ten.tea.2009.0226
21. Turner N J, Yates A J J, Weber D J, Qureshi I R, Stolz D B, Gilbert T W, Badylak S F. Xenogeneic extracellular matrix as an inductive scaffold for regeneration of a functioning musculotendinous junction. Tissue engineering. Part A. 2010; 16(11):3309-3317. doi:10.1089/ten.TEA.2010.0169
22. Lin C-H, Yang J-R, Chiang N-J, Ma H, Tsay R-Y. Evaluation of Decellularized Extracellular Matrix of Skeletal Muscle for Tissue Engineering. The International Journal of Artificial Organs. 2014; 37(7):546-555. doi:10.5301/ijao.5000344
23. Corona B T, Garg K, Ward C L, McDaniel J S, Walters T J, Rathbone C R. Autologous minced muscle grafts: a tissue engineering therapy for the volumetric loss of skeletal muscle. American Journal of Physiology-Cell Physiology. 2013; 305(7):C761-C775. doi:10.1152/ajpcell.00189.2013
24. Merritt E K, Cannon M V, Hammers D W, Le L N, Gokhale R, Sarathy A, Song T J, Tierney M T, Suggs L J, Walters T J, et al. Repair of Traumatic Skeletal Muscle Injury with Bone-Marrow-Derived Mesenchymal Stem Cells Seeded on Extracellular Matrix. Tissue Engineering Part A. 2010; 16(9):2871-2881. doi:10.1089/ten.tea.2009.0826
25. Coppi P De, Bellini S, Conconi M T, Sabatti M, Simonato E, Gamba P G, Nussdorfer G G, Parnigotto P P. Myoblast—Acellular Skeletal Muscle Matrix Constructs Guarantee a Long-Term Repair of Experimental Full-Thickness Abdominal Wall Defects. Tissue Engineering. 2006; 12(7):1929-1936. doi:10.1089/ten.2006.12.1929
26. Passipieri J A, Hu X, Mintz E, Dienes J, Baker H B, Wallace C H, Blemker S S, Christ G J. In Silico and In Vivo Studies Detect Functional Repair Mechanisms in a Volumetric Muscle Loss Injury. Tissue Engineering Part A. 2019 March 18. doi:10.1089/ten.tea.2018.0280
27. Kim J H, Seol Y-J, Ko I K, Kang H-W, Lee Y K, Yoo J J, Atala A, Lee S J. 3D Bioprinted Human Skeletal Muscle Constructs for Muscle Function Restoration. Scientific Reports. 2018; 8(1):12307. doi:10.1038/s41598-018-29968-5
28. Badylak S F. The extracellular matrix as a scaffold for tissue reconstruction. Seminars in cell & developmental biology. 2002; 13(5):377-383.
29. Brown B N, Badylak S F. Extracellular matrix as an inductive scaffold for functional tissue reconstruction. Translational Research. 2014; 163(4):268-285. doi:https://doi.org/10.1016/j.trs1.2013.11.003
30. Londono R, Badylak S F. Biologic scaffolds for regenerative medicine: mechanisms of in vivo remodeling. Annals of biomedical engineering. 2015; 43(3):577-592. doi:10.1007/s10439-014-1103-8
31. Cheng C W, Solorio L D, Alsberg E. Decellularized tissue and cell-derived extracellular matrices as scaffolds for orthopaedic tissue engineering. Biotechnology advances. 2014; 32(2):462-484. doi:10.1016/j.biotechadv.2013.12.012
32. Fuoco C, Petrilli L L, Cannata S, Gargioli C. Matrix scaffolding for stem cell guidance toward skeletal muscle tissue engineering. Journal of orthopaedic surgery and research. 2016; 11(1):86. doi:10.1186/s13018-016-0421-y
33. Theocharis A D, Skandalis S S, Gialeli C, Karamanos N K. Extracellular matrix structure. Advanced Drug Delivery Reviews. 2016; 97:4-27. doi:https://doi.org/10.1016/j.addr.2015.11.001
34. Badylak S F, Freytes D O, Gilbert T W. Extracellular matrix as a biological scaffold material: Structure and function. Acta biomaterialia. 2009; 5(1):1-13. doi:10.1016/j.actbio.2008.09.013
35. Badylak S F. Decellularized Allogeneic and Xenogeneic Tissue as a Bioscaffold for Regenerative Medicine: Factors that Influence the Host Response. Annals of Biomedical Engineering. 2014; 42(7):1517-1527. doi:10.1007/s10439-013-0963-7
36. Yi S, Ding F, Gu L G and X. Extracellular Matrix Scaffolds for Tissue Engineering and Regenerative Medicine. Current Stem Cell Research & Therapy. 2017; 12(3):233-246. doi:http://dx.doi.org/10.2174/1574888X11666160905092513
37. Choi J S, Kim J D, Yoon H S, Cho Y W. Full-thickness skin wound healing using human placenta-derived extracellular matrix containing bioactive molecules. Tissue engineering. Part A. 2013; 19(3-4):329-339. doi:10.1089/ten.TEA.2011.0738
38. Teodori L, Costa A, Marzio R, Perniconi B, Coletti D, Adamo S, Gupta B, Tarnok A. Native extracellular matrix: a new scaffolding platform for repair of damaged muscle. Frontiers in physiology. 2014; 5:218. doi:10.3389/fphys.2014.00218
39. Bernard M P, Chu M L, Myers J C, Ramirez F, Eikenberry E F, Prockop D J. Nucleotide sequences of complementary deoxyribonucleic acids for the pro alpha 1 chain of human type I procollagen. Statistical evaluation of structures that are conserved during evolution. Biochemistry. 1983; 22(22):5213-5223.
40. Groll J, Boland T, Blunk T, Burdick J A, Cho D-W, Dalton P D, Derby B, Forgacs G, Li Q, Mironov V A, et al. Biofabrication: reappraising the definition of an evolving field. Biofabrication. 2016; 8(1):13001. doi:10.1088/1758-5090/8/1/013001
41. Boland T, Mironov V, Gutowska A, Roth E A, Markwald R R. Cell and organ printing 2: Fusion of cell aggregates in three-dimensional gels. The Anatomical Record Part A: Discoveries in Molecular, Cellular, and Evolutionary Biology. 2003; 272A(2):497-502. doi:10.1002/ar.a.10059
42. Jakab K, Neagu A, Mironov V, Markwald R R, Forgacs G. Engineering biological structures of prescribed shape using self-assembling multicellular systems. Proceedings of the National Academy of Sciences of the United States of America. 2004; 101(9):2864 LP-2869. doi:10.1073/pnas.0400164101
43. Jakab K, Norotte C, Marga F, Murphy K, Vunjak-Novakovic G, Forgacs G. Tissue engineering by self-assembly and bio-printing of living cells. Biofabrication. 2010; 2(2):22001. doi:10.1088/1758-5082/2/2/022001
44. Mironov V, Visconti R P, Kasyanov V, Forgacs G, Drake C J, Markwald R R. Organ printing: Tissue spheroids as building blocks. Biomaterials. 2009; 30(12):2164-2174. doi:https://doi.org/10.1016/j.biomaterials.2008.12.084
45. Dicker K T, Gurski L A, Pradhan-Bhatt S, Witt R L, Farach-Carson M C, Jia X. Hyaluronan: A simple polysaccharide with diverse biological functions. Acta Biomaterialia. 2014; 10(4):1558-1570. doi:https://doi.org/10.1016/j.actbio.2013.12.019
46. Highley C B, Prestwich G D, Burdick J A. Recent advances in hyaluronic acid hydrogels for biomedical applications. Current Opinion in Biotechnology. 2016; 40:35-40. doi:https://doi.org/10.1016/j.copbio.2016.02.008
47. Allison D D, Grande-Allen K J. Review. Hyaluronan: A Powerful Tissue Engineering Tool. Tissue Engineering. 2006; 12(8):2131-2140. doi:10.1089/ten.2006.12.2131
48. Kang H-W, Lee S J, Ko 1K, Kengla C, Yoo J J, Atala A. A 3D bioprinting system to produce human-scale tissue constructs with structural integrity. Nature Biotechnology. 2016; 34:312.
49. Kolesky D B, Homan K A, Skylar-Scott M A, Lewis J A. Three-dimensional bioprinting of thick vascularized tissues. Proceedings of the National Academy of Sciences. 2016; 113(12):3179 LP-3184. doi:10.1073/pnas.1521342113
50. Huang S, Yao B, Xie J, Fu X. 3D bioprinted extracellular matrix mimics facilitate directed differentiation of epithelial progenitors for sweat gland regeneration. Acta Biomaterialia. 2016; 32:170-177. doi:https://doi.org/10.1016/j.actbio.2015.12.039
51. Yang X, Lu Z, Wu H, Li W, Zheng L, Zhao J. Collagen-alginate as bioink for three-dimensional (3D) cell printing based cartilage tissue engineering. Materials science & engineering. C, Materials for biological applications. 2018; 83:195-201. doi:10.1016/j.msec.2017.09.002
52. Kim J H, Yoo J J, Lee S J. Three-dimensional cell-based bioprinting for soft tissue regeneration. Tissue Engineering and Regenerative Medicine. 2016; 13(6):647-662. doi:10.1007/s13770-016-0133-8
53. Jang J, Park H-J, Kim S-W, Kim H, Park J Y, Na S J, Kim H J, Park M N, Choi S H, Park S H, et al. 3D printed complex tissue construct using stem cell-laden decellularized extracellular matrix bioinks for cardiac repair. Biomaterials. 2017; 112:264-274. doi:https://doi.org/10.1016/j.biomaterials.2016.10.026
54. Kim B S, Kim H, Gao G, Jang J, Cho D-W. Decellularized extracellular matrix: a step towards the next generation source for bioink manufacturing. Biofabrication. 2017; 9(3):034104. doi:10.1088/1758-5090/aa7e98.

### ADDITIONAL REFERENCES

The devices, systems, apparatuses, compositions, materials, machine readable medium, computer program products, and methods of various embodiments of the invention disclosed herein may utilize aspects (such as devices, systems, apparatuses, compositions, materials, machine readable medium, computer program products, and methods) disclosed in the following references, applications, publications and patents and which are hereby incorporated by reference herein in their entirety, and which are not admitted to be prior art with respect to the present invention by inclusion in this section:
A. Sill, T. J., & von Recum, H. A. (2008). Electrospinning: Applications in drug delivery and tissue engineering. Biomaterials, 29(13), 1989-2006. doi:10.1016/j.biomaterials.2008.01.011
B. Drury, J. L., & Mooney, D. J. (2003). Hydrogels for tissue engineering: Scaffold design variables and applications. Biomaterials, 24(24), 4337-4351. doi:10.1016/S0142-9612(03)00340-5
C. Li, W. -., Laurencin, C. T., Caterson, E. J., Tuan, R. S., & Ko, F. K. (2002). Electrospun nanofibrous structure: A novel scaffold for tissue engineering. Journal of Biomedical Materials Research, 60(4), 613-621. doi:10.1002/jbm.10167
D. Matthews, J. A., Wnek, G. E., Simpson, D. G., & Bowlin, G. L. (2002). Electrospinning of collagen nanofibers. Biomacromolecules, 3(2), 232-238. doi:10.1021/bm015533u
E. Sisson, K., Zhang, C., Farach-Carson, M. C., Chase, D. B., & Rabolt, J. F. (2009). Evaluation of cross-linking methods for electrospun gelatin on cell growth and viability. Biomacromolecules, 10(7), 1675-1680. doi:10.1021/bm900036s
F. Bischel, L. L., Coneski, P. N., Lundin, J. G., Wu, P. K., Giller, C. B., Wynne, J., Ringeisen, B. R., Pirlo, R. K. (2016). Electrospun gelatin biopapers as substrate for in vitro bilayer models of blood-brain barrier tissue. Journal of Biomedical Materials Research—Part A, 104(4), 901-909. doi:10.1002/jbm.a.35624.
G. Nguyen, D. G. et al. Bioprinted 3D Primary Liver Tissues Allow Assessment of Organ-Level Response to Clinical Drug Induced Toxicity In Vitro. PLoS One 11, e0158674, doi:10.1371/journal.pone.0158674 (2016).
H. Nguyen, D. G. & Pentoney, S. L., Jr. Bioprinted three dimensional human tissues for toxicology and disease modeling. Drug Discov Today Technol 23, 37-44, doi:10.1016/j.ddtec.2017.03.001 (2017).
I. Norona, L. M., Nguyen, D. G., Gerber, D. A., Presnell, S. C. & LeCluyse, E. L. Editor's Highlight: Modeling Compound-Induced Fibrogenesis In Vitro Using Three-Dimensional Bioprinted Human Liver Tissues. Toxicol Sci 154, 354-367, doi:10.1093/toxsci/kfw169 (2016).
J. King, S. M. et al. 3D Proximal Tubule Tissues Recapitulate Key Aspects of Renal Physiology to Enable Nephrotoxicity Testing. Front Physiol 8, 123, doi:10.3389/fphys.2017.00123 (2017).
K. U.S. Utility patent application Ser. No. 15/760,009, entitled “BIOREACTOR AND RESEEDING CHAMBER SYSTEM AND RELATED METHODS THEREOF”, filed Mar. 14, 2018; Publication No. US-2018-0265831-A1, Sep. 20, 2018.
L. International Patent Application Serial No. PCT/US2017/045299, entitled “BIOREACTOR CONTROLLER DEVICE AND RELATED METHOD THEREOF”, filed Aug. 3, 2017; Publication No. WO 2018/027033, Feb. 8, 2018.
M. International Patent Application Serial No. PCT/US2016/051948, entitled “BIOREACTOR AND RESEEDING CHAMBER SYSTEM AND RELATED METHODS THEREOF”, filed Sep. 15, 2016; Publication No. WO 2017/048961, Mar. 23, 2017.
N. U.S. Pat. No. 9,493,735 B2, Yoo, et al., “Bioreactor System and Method of Enhancing Functionality of Muscle Cultured in Vitro”, Nov. 15, 2016. 0. U.S. Pat. No. 9,506,025 B2, Yoo, et al., “Cultured Muscle Produced by Mechanical Conditioning”, Nov. 29, 2016.
P. U.S. Pat. No. 9,556,418 B2, Christ, et al., “Methods for Making a Tissue Engineered Muscle Repair (TEMR) Construction in Vitro for Implantation in Vivo”, Jan. 31, 2017.
Q. U.S. Pat. No. 9,757,225 B2, Yoo, et al., “Bioreactor System and Method of Enhancing Functionality of Muscle Cultured in Vitro”, Sep. 12, 2017.
R. U.S. Patent Application Publication No. US 2012/0100602 A1 to Lu, et al., “Bioreactor System for Mechanical Stimulation of Biological Samples”, Apr. 26, 2012.
S. U.S. Patent Application Publication No. US 2011/0172683 A1 to Yoo, et al., “Tissue Expander”, Jul. 14, 2011.
T. U.S. Patent Application Publication No. US 2006/0239981 A1 to Yoo, et al., “Bioreactor System and Method of Enhancing Functionality of Muscle Cultured in Vitro”, Oct. 26, 2006.
U. U.S. Patent Application Publication No. US 2006/0141623 A1 to Smith, et al., “Automated Tissue Engineering System”, Jun. 29, 2006.
V. U.S. Patent Application Publication No. US 2011/0212500 A1 to Boronyak, et al, “Flow-Stretch-Flexure Bioreactor”, Sep. 1, 2011.
W. U.S. Patent Application Publication No. US 2004/0219659 A1 to Altman, et al., “Multi-Dimensional Strain Bioreactor”, Nov. 4, 2004.
X. U.S. Patent Application Publication No. 2009/0265005 A1 to Yoo, et al., “Bioreactor System and Method of Enhancing Functionality of Muscle Cultured in Vitro”, Oct. 22, 2009.
Y. U.S. Pat. No. 5,795,710 to Park, “Method and Apparatus for Organ Culture”, Aug. 18, 1998.
Z. International Patent Application Publication No. WO 2016/036764 A2 to Bonvillain, et al, “Automated Bioreactor System, System for Automatically Implementing Protocol for Decellularizing Organ, and Waste Decontamination System, Mar. 10, 2016.
AA. U.S. Patent Application Publication No. US 2012/0086657 A1 to Stanton, I V, et al., “Configurable Methods and Systems of Growing and Harvesting Cells in a Hollow Fiber Bioreactor System”, Apr. 12, 2012.
BB. Chinese Patent Application Publication No. CN100525063 to THK Co., Ltd, “Error Detection Method and Motor Control Device”, Aug. 5, 2009.
CC. U.S. Pat. No. 7,439,693 B2 to Shoda, et al., “Anomaly Detection Method and Motor Control Device”, Oct. 21, 2008.
DD. U.S. Utility patent application Ser. No. 16/322,691 to Christ, et al, “Bioreactor Controller Device and Related Method Thereof”, Feb. 1, 2019

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A bioprinting method, said method comprising:
disposing a scaffold onto a bioassembly device;
disposing said bioassembly device, with said scaffold, onto a bioprinter;
bioprinting onto a first side of said scaffold or both said first side and a second side of said scaffold, which is disposed on said bioassembly device that is disposed on said bioprinter;
transferring said bioprinted scaffold, which is disposed on said bioassembly device, onto a bioreactor; and
creating tissue engineered construct while said bioprinted scaffold remains on said bioassembly device and in said bioreactor.

2. The method of claim 1, wherein said scaffold comprises a sheet-based scaffold.

3. The method of claim 1, wherein said tissue engineered construct comprises at least one or more of any combination of the following:
implantable tissue engineered construct;
three dimensional structure tissue engineered construct;
solid organs construct;
organoids construct;
sheet-like construct;
varying geometrical shapes of said construct; and
distinct consistency on a first side of said contrast relative to a second side of said construct.

4. The method of claim 3, further comprising:
folding said sheet-like construct.

5. The method of claim 3, further comprising:
repeating steps of claim 1 one or more times, and stacking two or more of said constructs.

6. The method of claim 1, wherein said bioprinting includes directly depositing cells onto said first side of said scaffold or both said first side and a second side of said scaffold.

7. The method of claim 6, wherein said bioprinting comprises encapsulating said cells being depositing in a gel.

8. The method of claim 6, wherein said bioprinting comprises controlling the number of cells being deposited and/or type of cells being deposited.

9. The method of claim 1, wherein said bioprinting includes extruding bioink onto said first side of said scaffold or both said first side and a second side of said scaffold.

10. The method of claim 9, wherein said bioink comprises at least one or more of any combination of the following: hyaluronic acid (HA), gelatin, alginate, fibrinogen, collagen, and other biopolymers.

11. The method of claim 1, wherein said creating comprises: culturing, differentiating, and preconditioning said scaffold in said bioreactor while said scaffold remains on said bioassembly device.

12. The method of claim 1, wherein said creating comprises:
incubating said bioprinted scaffold.

13. The method of claim 11, wherein said creating comprises:
stretching said bioprinted scaffold.

14. The method of claim 1, wherein said creating comprises:
seeding said first side of said bioprinted scaffold or both said first side and a second side of said bioprinted scaffold.

15. The method of claim 14, wherein said seeding includes controlling cell seeding density and/or cell seeding consistency.

16. The method of claim 1, wherein said disposing said scaffold onto said bioassembly device includes securing said scaffold in position for said bioprinting.

17. The method of claim 1, wherein said disposing said scaffold onto said bioassembly device includes securing said scaffold in a taut position for said bioprinting.

18. The method of claim 17, wherein disposing said bioassembly device includes securing said bioassembly device to said bioprinter.

19. The method of claim 18, wherein said securing said bioassembly device to said bioprinter comprises disposing a plate on said bioprinter configured to receive said bioassembly device.

20. The method of claim 18, wherein after transferring said bioprinted scaffold that is disposed on said bioassembly device, securing said bioassembly device to said bioreactor.

21. The method of claim 20, wherein said disposing said scaffold onto said bioassembly device includes securing said scaffold in a taut position while in said bioreactor.

22. A bioassembly device for use with a bioprinter, said device comprising:
a top portion and a bottom portion that are configured to secure a scaffold there between while said bioprinter performs bioprinting onto a first side of said scaffold or both said first side and a second side of said scaffold.

23. The device of claim 22, wherein said top portion and said bottom portion are configured to secure said bioprinted scaffold while it is transferred to a bioreactor.

24. The device of claim 22, wherein said top portion and said bottom portion are configured to:
slidably connect together with one another; or
snap-fit connect with one another one another.

25. The device of claim 23, wherein said top portion and said bottom portion are configured to secure said transferred bioprinted scaffold in said bioreactor while said scaffold is created into tissue engineered construct.

26. The device of claim 25 provided in a kit, wherein said kit includes said scaffold.

27. The device of claim 26, wherein said kit provides said scaffold as said tissue engineered construct that comprises at least one or more of any combination of the following:
implantable tissue engineered construct;
three-dimensional structure tissue engineered construct;
solid organs construct;
organoids construct;
sheet-like construct;
varying geometrical shapes of said construct; and
distinct consistency on a first side of said contrast relative to a second side of said construct.

28. The device of claim 26, wherein said kit provides said scaffold in a folded configuration construct.

29. The device of claim 26, wherein said kit provides two or more said scaffolds wherein said two or more said scaffolds are stacked to form said construct.

30. The device of claim 22, wherein said top portion and said bottom portion are configured to secure said scaffold there between while cells are deposited onto said first side of said scaffold or both said first side and a second side of said scaffold during said bioprinting.

31. The device of claim 30, wherein said top portion and said bottom portion are configured to secure said scaffold there between while said cells are encapsulated in a gel during bioprinting.

32. The device of claim 22, wherein said top portion and said bottom portion that are configured to secure said scaffold comprises at least one or more of the following:
a frame configured to provide the scaffold securement;
a portion of a frame configured to provide the scaffold securement;
a clamp configured to provide the scaffold securement; or
bars or elongated members arranged to provide the scaffold securement.

33. The device of claim 22, wherein said securing said scaffold while in said bioprinter includes securing said scaffold in a taut position for said bioprinting.

34. The device of claim 22, wherein said top portion and said bottom portion are configured to be secured in place at a designated location in said bioprinter.

35. The device of claim 23, wherein said top portion and bottom portion are configured to be secured in place at a designated location in said bioreactor transferred therein.

36. The device of claim 23, wherein:
said securing said scaffold while in said bioprinter includes securing said scaffold in a taut position for said bioprinting; and
said securing said scaffold while in said bioreactor includes securing said scaffold in a taut position while in said bioreactor.

37. The device of claim 22 provided in a kit, wherein said kit includes said bioprinter.

38. The device of claim 23 provided in a kit, wherein said kit includes said bioprinter and said bioreactor.

39. A bioprinting system, said system comprising:
a designated area configured for receiving a bioassembly device, which includes a scaffold disposed in said bioassembly device; and
a print head configured for bioprinting onto a first side of said scaffold or both said first side and a second side of said scaffold, while said bioassembly device is in said designated area of said bioprinting system.

40. The system of claim 39, wherein said bioprinting includes directly depositing cells onto said first side of said scaffold or both said first side and a second side of said scaffold.

41. The system of claim 40, wherein said bioprinting comprises encapsulating said cells being depositing in a gel.

42. The system of claim 40, wherein said bioprinting comprises controlling the number of cells being deposited and/or type of cells being deposited.

43. The system of claim 39, wherein said bioprinting includes extruding bioink onto said first side of said scaffold or both said first side and a second side of said scaffold.

44. The system of claim 39, wherein said designated area is configured to secure said bioassembly device to said bioprinting system.

45. The system of claim 39, further comprising a kit, wherein said system may be provided with a bioreactor, and wherein said bioassembly device is configured to secure said bioprinted scaffold while it is transferred to said bioreactor.

46. The system of claim 45, further comprising a kit, wherein said system may be provided with a bioreactor, and wherein said bioassembly device is configured to secure said bioprinted scaffold at a designated location in said bioreactor transferred therein.
